(19) United States
(12) Patent Application Publication
(10) Pub. No.: US 2003/0092010 A1
(43) Pub. Date: May 15, 2003

(54) Molecular switches

(75) Inventors: Yen Choo, Cambridge (GB); Christopher Graeme Ullman, London (GB)

Correspondence Address: ROBINS & PASTERNAK LLP, 545 MIDDLEFIELD ROAD, SUITE 180, MENLO PARK, CA, 94025, US

(73) Assignee: Sangamo BioSciences, Inc., Richmond, CA

(21) Appl. No.: 09/996,484

(22) Filed: Nov. 28, 2001

(30) Foreign Application Priority Data
May 30, 2000 (GB) PCT/GB00/02080
May 28, 1999 (GB) 9912635.1
Jan. 24, 2000 (GB) 0001582.6

(51) Int. Cl. C12Q 1/68; G01N 33/53

(52) U.S. Cl. 435/6; 435/7.1

(57) ABSTRACT

Provided herein are compositions comprising molecular switches and methods for identifying, selecting and using such molecular switches. Also provided are methods for the identification and use of ligand-dependent binding molecules and ligands.

### FIELD OF THE INVENTION

This invention relates to molecular switches and methods for identifying and selecting such switches. Particular molecular switches include gene switches that use molecules capable of binding a specific DNA sequence in a ligand-dependent manner, and protein switches in which two protein binding partners bind in a manner which is modulatable by a ligand. Moreover, this invention relates to methods for the identification of the ligand-dependent binding molecules as well as identification of ligands.

### BACKGROUND TO THE INVENTION

Protein-protein interactions are crucial to almost every physiological and pharmacological process. These interactions often are characterized by very high affinity, with dissociation constants in the low nanomolar to subpicomolar range. Such strong affinity between proteins is possible when a high level of specificity allows subtle discrimination among closely related structures. Proteins can bind to each other through several types of interface, for example, a “surface string” where a portion of the surface of one protein contacts an extended loop of polypeptide chain on a second protein, a “helix-helix” configuration involving two alpha helices, and a “surface-surface” configuration involving the matching of one surface to another. For example, it is known that the SH2 domain binds tightly to a region of a polypeptide chain that contains a phosphorylated tyrosine side chain.

Polypeptides can form higher order tertiary structures with like polypeptides (homo-oligomers) or with unalike polypeptides (hetero-oligomers). In the simplest scenario, two identical polypeptides associate to form an active homodimer. An example of this type of association is the natural association of myosin II molecules in the assembly of myosin into filaments. Protein-protein association may be mediated by several factors, including post-translational modifications, by means of which enzymatic activity may be biologically controlled. For example, the phosphorylation state of a protein may cause it to associate with or dissociate from another protein. The phospohnrylation state of a protein is thought to be determined by the relative activity of protein kinases which add phosphate and protein phosphatases which remove the phosphate moiety from the protein. For example, it is thought that phosphorylation of myosin II by protein kinases is involved in the priming event leading to dimerization of myosin II monomers and subsequent formation of myosin filaments.

Ligand mediated association and dissociation of proteins is also known, in which the ability of a protein to interact with another protein is dependent on the binding of a ligand to one or both proteins. An example of ligand-mediated heterodimer association is described in patent application number W092/00388. This publication describes an adenosine 3:5 cyclic monophosphate (cAMP) dependent protein kinase which is a four-subunit enzyme being composed of two catalytic polypeptides (C) and two regulatory polypeptides (R). In nature the polypeptides associate in a stoichiometry of R2C2. In the absence of cAMP the R and C subunits associate and the enzyme complex is inactive. In the presence of cAMP the R subunit functions as a ligand for cAMP resulting in dissociation of the complex and the release of active protein kinase. The invention described in W092/00388 exploits this association by adding fluorochromes to the R and C subunits.

Proteins can also interact with nucleic acids, for example. DNA. Zinc finger proteins are transcriptional regulators of gene expression which may be adapted to regulate a desired gene by modulating the binding specificity of the zinc finger for its target nucleic acid. A number of applications for zinc finger technology have been suggested, including the treatment of diseases, use as reagents for manipulating nucleic acids and the regulation of gene expression.

One of the drawbacks of zinc fingers is that they are relatively large polypeptides. In order to introduce zinc fingers into a cell, it is necessary either to express the zinc fingers in the cell by means of a transgene, or to modify them to include cellular uptake domains which successfully target the zinc finger to the nucleus. In either case, but particularly the former, regulation of zinc finger activity is difficult, because the amount of zinc finger present in each cell nucleus can only be controlled indirectly, by influencing the level of zinc finger expression, or by varying the amount of protein administered to the cell. In both cases, upregulation or downregulation of zinc finger activity is slow. Downregulation in particular is dependent on the natural turnover of zinc finger molecules within the cell.

A number of promoter systems are known which can be regulated by small molecules, which molecules act effectively as a gene switch and allow the gene to be switched on or off. The ability to apply gene switch capability to any desired promoter would be highly desirable. Most promoters of clinical or commercial significance, however, do not possess regulatory elements which are susceptible to gene switch regulation.

### SUMMARY OF THE INVENTION

According to a first aspect of the invention, we provide a method of selecting a switching system, the switching system comprising: (i) a first component comprising a first molecule and (ii) a second component comprising a second molecule, in which the first molecule binds to the second molecule in a manner modulatable by a ligand and (iii) a third component comprising the ligand, the method comprising the steps of. (a) contacting one or more candidate first molecules with one or more candidate second molecules in the presence of one or more ligands: (b) selecting a complex of the three components; (c) optionally isolating and/or identifying the unknown components of the complex: (d) comparing the binding of the first molecule component of the complex to the second molecule component of the complex in the presence and absence of the ligand component of the complex; and (e) selecting complexes where said binding differs in the presence and absence of the ligand component, in which at least one component is provided in the form of a library of members.

Preferably, at least one of the candidate first molecules comprises a non-naturally occurring binding domain which binds to the second molecule. The term “a non-naturally occurring binding domain” means that the binding domain does not occur in nature, even as part of a larger molecule, and has been obtained by deliberate mutagenesis procedures or de novo design techniques.

Our method may be used to select switches in which the interaction between a DNA and a protein is modulated by a ligand, in other words, a gene switch. In this embodiment. one of the first molecule and second molecule comprises a nucleic acid binding molecule. and the other of the first and second molecules comprises a nucleic acid.

Preferably, the nucleic acid binding molecules is provided as a plurality of nucleic acid binding molecules, more preferably the nucleic acid binding molecule is provided as a library of nucleic acid binding molecules. In a highly preferred embodiment, the candidate nucleic acid binding molecules are provided as a phage display library. Where only one nucleic acid binding molecule is included in the screen, the nucleic acid binding molecule preferably comprises a non-naturally occurring nucleic acid binding domain. The nucleic acid binding domain includes nucleic acid-binding residues and those residues which form part of the same polypeptide domain, but do not necessarily bind to the nucleic acid itself.

In the context of the present invention. “nucleic acid” includes DNA. RNA, or any other form of natural, partially or completely synthetic nucleic acids. Preferably the target nucleic acid is provided as a plurality of nucleic acid sequences, more preferably as a library of nucleic acid sequences, said sequences being related to one another by sequence homology.

In one embodiment, a plurality or library of candidate ligands are used, in which case it is preferred to use one target nucleic acid. However, the invention encompasses the simultaneous screening of two or three libraries, such that in a preferred embodiment all three components of the switching system are provided in the form of a library. As used herein, the term “library” refers to a plurality of individual members which vary in structure, such that the library includes a repertoire of component members. The term “library” as applied to nucleic acids may also refer to nucleic acids which encode a polypeptide repertoire. Variability may be introduced into nucleic acid libraries by any one of a variety of techniques, including the use of error-prone polymerases, mutator strains of host cells, chemical mutagenesis, radiation mutagenesis, and the like. Variation is introduced into chemical libraries by techniques known for use in combinatorial chemistry approaches.

Our method involves an optional step of isolating and/or identifying the unknown components of the complex. When applied to selecting a gene switch, these components may include a ligand component a nucleic acid binding molecule component, or a nucleic acid, or any combination of these. Preferably, the candidate nucleic acid binding molecules are polypeptides, which may be at least partly derived from DNA binding proteins. Preferably, these DNA binding proteins are transcription factors. Preferably, the candidate nucleic acid binding molecules are derived from zinc finger transcription factors. Preferably, the ligand is selected from Distamycin A, Actinomycin D and echinomycin.

Another application of our method is to select switches in which the interaction between a protein and a protein is modulated by a ligand. We refer to these switches as “protein switches”. In this embodiment, both the first and second molecules comprise a polypeptide. Preferably, one of the first and second molecules comprises a polypeptide binding molecule (which is preferably a polypeptide binding protein) and the other of the first and second molecules comprises a polypeptide. One or both of the polypeptide binding molecule and the polypeptide may additionally be capable of binding to a nucleic acid, for example, DNA. Preferably, the first molecule is a nucleic acid binding protein capable of binding to nucleic acid. In a highly preferred embodiment, the nucleic acid binding protein binds to nucleic acid in a manner modulatable by the second molecule. Thus, the nucleic acid binding protein may bind to or be released from the nucleic acid depending on whether the second (polypeptide) molecule is bound to it. As the nucleic acid binding protein binds to the second (polypeptide) molecule in a manner which is modulated by the ligand, the binding of the nucleic acid binding protein to the nucleic acid is ultimately modulated by the ligand in this embodiment.

One or both of the first and second polypeptide molecules may be provided as a plurality of polypeptides, preferably in the form of a library of polypeptides. More preferably, the ligand is an immunoglobulin molecule, preferably an antibody molecule. As with selection of gene switches described above, a plurality or library of candidate ligands may be used. Where a plurality or library of candidate ligands is used, the polypeptide components of the protein switch may be single known or unknown polypeptides, or alternatively the screening method involves a single first polypeptide, and the other provided as a library. Alternatively, all three components of the protein switch (i.e., protein. protein and ligand) may be provided in the form of libraries as defined above. Thus, the invention as it relates to selection of protein switches encompasses the simultaneous screening of two or three libraries. As with nucleic acid libraries, variability may be introduced into polypeptide or ligand libraries by means known in the art, for example, by mutagenising a corresponding nucleic acid library (as described above) which encodes members of the polypeptide library, or by chemical synthesis, etc.

In a preferred embodiment of the first aspect of the invention, the first molecule component of the complex has a higher affinity for the second molecule component of the complex in the presence of the ligand component than in the absence of the ligand component. Alternatively, the first molecule component of the complex has a higher affinity for the second molecule component of the complex in the absence of the ligand component than in the presence of the ligand component.

We provide, according to a second alternative aspect of the invention, a switching system comprising a gene switch, in which the switching system has been selected by a method according to the first aspect of the invention.

There is provided, according to a third alternative aspect of the invention, a nucleic acid binding molecule selected by a method according to the first aspect of the invention in a method of regulating transcription from a nucleic acid sequence comprising a target nucleic acid to which the nucleic acid binding molecule binds in a manner modulatable by a ligand. We also provide a polypeptide binding molecule selected by a method according to the first aspect of the invention in a method of regulating transcription from a nucleic acid sequence comprising a target nucleic acid sequence, in which the polypeptide binding molecule interacts with a polypeptide to bind the nucleic acid target sequence, the interaction being in a manner modulatable by a ligand.

Furthermore, we provide, according to a fourth alternative aspect of the invention, a ligand selected by a method according to the first aspect of the invention in a method of regulating transcription from a nucleic acid sequence comprising a target nucleic acid to which a nucleic acid binding molecule binds in a manner modulatable by the ligand.

There is provided according to a fifth alternative aspect of the invention, use of a target nucleic acid selected by the method according to the first aspect of the invention in a method of regulating transcription from a nucleic acid sequence comprising the target nucleic acid to which a nucleic acid binding molecule binds in a manner modulatable by a ligand.

According to a sixth alternative aspect of the invention, there is provided a method of modulating the expression of one or more genes, said method comprising administering a nucleic acid binding molecule and a ligand selected according to a method according to the first aspect of the invention to a cell, in which the regulatory sequences of the genes comprise a target nucleic acid selected according to a method according to the first aspect of the invention.

We provide according to a seventh alternative aspect of the invention, a method of modulating the expression of one or more nucleotide sequences of interest in a host cell which host cell comprises a nucleic acid sequence capable of directing the expression of a nucleic acid binding molecule and a target nucleic acid sequence to which the nucleic acid binding molecule binds in a manner modulatable by a ligand, which method comprises administering said ligand to the cell and wherein the nucleic acid binding molecule is heterologous to the host cell.

Preferably, the host cell is a plant cell. More preferably, the plant cell is part of a plant and the target sequence is part of a regulatory sequence to which the nucleotide sequence of interest is operably linked, said regulatory sequence being preferentially active in the male or female organs of the plant.

According to an eighth alternative aspect of the invention, there is provided a non human transgenic organism comprising a target nucleic acid sequence and a nucleic acid sequence capable of directing the expression of a nucleic acid binding molecule which binds to the target nucleic acid in a manner modulatable by a ligand, in which the target nucleic acid sequence and/or nucleic acid sequence are heterologous to the organism.

There is provided, according to a ninth alternative aspect of the invention, a transgenic non-human organism according to the eighth aspect which is a plant.

We provide, according to a tenth alternative aspect of the invention, a switching system comprising a protein switch, in which the switching system has been selected by a method according to the first aspect, in which each of the first and second molecules comprises a polypeptide. Preferred features of the tenth aspect include: the first molecule is a nucleic acid binding protein capable of binding to a nucleic acid, and the nucleic acid binding protein binds to nucleic acid in a manner modulatable by the second molecule.

There is provided, according to an eleventh aspect of the invention, use of a nucleic acid binding protein selected by a method according to the tenth aspect with said preferred features, in a method of regulating transcription from a nucleic acid sequence comprising a target nucleic acid to which the nucleic acid binding protein binds. We provide, according to a twelfth aspect of the invention, use of a ligand selected by a method according to the tenth aspect with said preferred features, in a method of regulating transcription from a nucleic acid sequence comprising a target nucleic acid to which the nucleic acid binding protein binds in a manner modulatable by the ligand. There is provided, according to a 13^{th} aspect of the invention, use of a target nucleic acid selected by the method according to he tenth aspect with said preferred features, in a method of regulating transcription from a nucleic acid sequence comprising a target nucleic acid to which the nucleic acid binding protein binds in a manner modulatable by a ligand. We further provide a 14^{th} alternative aspect a method of modulating the expression of one or more genes, said method comprising administering a nucleic acid binding protein and a ligand selected according to a method according to he tenth aspect with said preferred features to a cell, in which the regulatory sequences of the genes comprise a target nucleic acid to which the nucleic acid binding protein binds in a manner modulatable by a ligand.

Further aspects include a 15^{th} alternative aspect, which is a method of modulating the expression of one or more nucleotide sequences of interest in a host cell which host cell comprises a first nucleic acid sequence capable of directing the expression of a nucleic acid binding protein, a second nucleic acid sequence capable of directing the expression of a second polypeptide, the binding between the nucleic acid binding to the second polypeptide being modulatable by a ligand, and a target nucleic acid sequence to which the nucleic acid binding protein binds in a manner modulatable by a second polypeptide, which method comprises administering said ligand to the cell. Preferably, the nucleic acid binding protein is heterologous to the host cell. More preferably, the host cell is a plant cell. We also provide such a method in which the plant cell is part of a plant and the target sequence is part of a regulatory sequence to which the nucleotide sequence of interest is operably linked, said regulatory sequence being preferentially active in the male or female organs of the plant.

There is also provided according to a further aspect of the invention, a non human transgenic organism comprising a target nucleic acid sequence, a first nucleic acid sequence capable of directing the expression of a nucleic acid binding protein, and a second nucleic acid sequence capable of directing the expression of a second polypeptide which binds to the nucleic acid binding protein in a manner modulatable by a ligand, in which the nucleic acid binding protein binds to the target nucleic acid sequence in a manner modulatable by binding of the second polypeptide.

### DETAILED DESCRIPTION OF THE INVENTION

Unless defined otherwise, all technical and scientific terms used herein have the same meaning as commonly understood by one of ordinary skill in the art (e.g. in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual. 2d ed. (1989) Cold Spring Harbor Laboratory Press, Cold Spring Harbor, N.Y., and Ausubel et al, Short Protocols in Molecular Biology (1999) 4^{th} Ed, John Wiley & Sons, Inc. which are incorporated herein by reference), chemical methods, pharmaceutical formulations and delivery and treatment of patients.

The term ‘modulatable by’ is used to indicate that binding of the first molecule to the second molecule can be modulated or affected by the ligand. As applied to a gene switch, therefore, the ligand modulates or affects the binding of the nucleic acid binding molecule to the nucleic acid, and (as applied to a protein switch), the binding of the two polypeptide molecules is modulated or affected by the ligand. In other words, the ligand can modulate, affect, regulate, adjust, alter, or vary the binding of the first molecule to the second molecule.

The term ‘isolating’ in the context of the invention, refers to the act of removing one or more components or molecules from a sample of candidate molecules which are used in the methods disclosed herein.

The term ‘complex’ is used to describe an association between a DNA and one or more molecules as defined herein, or between a polypeptide molecule and one or more molecules. In the case of a polypeptide, these molecules may include another polypeptide molecule and/or a ligand molecule.

The term “gene switch” is used herein to describe a multiple component system comprising (i) a target DNA molecule; (ii) a DNA binding molecule which binds to the target DNA molecule in a manner modulatable by a ligand; and (iii) the ligand. The DNA binding molecule may or may not comprise a transcriptional effector domain, especially when part of the assay procedure. However, since ultimately the gene switch will be used to regulate transcription from one or more promoters, the DNA binding molecule may need to be modified to include a transcriptional activator or repressor domain, if one is not already present.

The term “protein switch” is used herein to describe a multiple component system comprising (i) a first polypeptide molecule; (ii) a second polypeptide molecule which binds to the first polypeptide molecule in a manner modulatable by a ligand; and (iii) the ligand.

A. Nucleic Acid Binding Molecules and Polypeptides

The term “nucleic acid binding molecule” includes any molecule which is capable of binding or associating with nucleic acid. This binding or association may be via covalent bonding, via ionic bonding, via hydrogen bonding, via Van-der-Waals bonding, or via any other type of reversible or irreversible association. In the context of the present invention. “nucleic acid” is usually DNA. However it may be RNA, as set out below, or any other form of nucleic acid, including completely or partially synthetic nucleic acids. Reference to ‘nucleic acid binding molecule’ is to be taken to include reference to “DNA binding molecule”. which term is to be construed accordingly as referring to any molecule which can bind to DNA.

As used herein the terms “peptide”, “polypeptide” and “protein” refer to a polymer in which the monomers are amino acids and are joined together through peptide or disulfide bonds. “Polypeptide” refers to either a full-length naturally-occurring amino acid chain or a “fragment thereof” or “peptide”, such as a selected region of the polypeptide that binds to another protein, peptide or polypeptide in a manner modulatable by a ligand, or to an amino acid polymer, or a fragment or peptide thereof, which is partially or wholly non-natural. “Fragment thereof” thus refers to an amino acid sequence that is a portion of a full-length polypeptide, between about 8 and about 500 amino acids in length, preferably about 8 to about 300, more preferably about 8 to about 200 amino acids, and even more preferably about 10 to about 50 or 100 amino acids in length. “Peptide” refers to a short amino acid sequence that is 1040 amino acids long, preferably 10-35 amino acids. Additionally, unnatural amino acids, for example, β-alanine, phenyl glycine and homoarginine may be included. Commonly-encountered amino acids which are not gene-encoded may also be used in the present invention. All of the amino acids used in the present invention may be either the D- or L-optical isomer. The L-isomers are preferred. In addition, other peptidomimetics are also useful, e.g. in linker sequences of polypeptides of the present invention (see Spatola, 1983. in Chemistry and Biochemistry of Amino Acids, Peiptides and Proteins, Weinstein, ed., Marcel Dekker, New York, p. 267). A “polypeptide binding molecule” is a molecule, preferably a polypeptide, protein or peptide, which has the ability to bind to another polypeptide, protein or peptide. Preferably, this binding ability is modulatable by a ligand.

The term “synthetic”, as used herein, is defined as that which is produced by in vitro chemical.

As used herein, the term “domain” refers to a linear sequence of amino acids which exhibits biological function, such as the ability to bind another molecule (for example, another polypeptide or fragment thereof). This linear sequence includes full-length amino acid sequences (e.g., those encoded by a full-length gene or polynucleotide), or a portion or fragment thereof, provided the biological function, in particular binding ability, is maintained by that portion or fragment. The term “domain” also may refer to polypeptides and peptides having biological function. A polypeptide useful in the invention will at least have a binding capability, i.e., with respect to binding as or to a binding partner, and also may have another biological function that is a biological function of a protein or domain from which the peptide sequence is derived.

The term ‘molecule’ is used herein to refer to any atom, ion, molecule, macromolecule (for example polypeptide), or combination of such entities. The term ‘ligand’ is used interchangeably with the term ‘molecule’. Molecules according the invention may be free in solution, or may be partially or fully immobilised. They may be present as discrete entities, or may be complexed with other molecules. Preferably, molecules according to the invention include polypeptides displayed on the surface of bacteriophage particles. More preferably, molecules according to the invention include libraries of polypeptides presented as integral parts of the envelope proteins on the outer surface of bacteriophage particles. Methods for the production of libraries encoding randomised polypeptides are known in the art and may be applied in the present invention. Randomisation may be total, or partial; in the case of partial randomisation, the selected codons preferably encode options for amino acids, and not for stop codons.

The term ‘candidate nucleic binding molecules’ is used to describe any one or more molecule(s) as defined above which may or may not be capable of binding nucleic acids. The capability of said molecules to bind nucleic acids may or may not be modulatable by a ligand. The latter of these properties may be investigated by the methods of this invention. Preferably, candidate nucleic acid binding molecules such as DNA binding molecules comprise a plurality of, or a library of polypeptides. More preferably, these polypeptides are, or are derived from, nucleic acid binding proteins (including DNA binding proteins) such as DNA repair enzymes, polymerases, recombinases, methylases, restriction enzymes. replication factors, histones, or DNA binding structural proteins such as chromosomal scaffold proteins; even more preferably said polypeptides are derived from DNA binding proteins, preferably transcription factors. ‘Derived from’ means that the candidate binding molecules preferably comprise one or more of; DNA binding proteins, transcription factors, fragment(s) of DNA binding proteins or transcription factors, sequences homologous to DNA binding proteins or transcription factors, or polypeptides which have been fully or partially randomised from a starting sequence which is a DNA binding proteins or a transcription factor, a fragment of a DNA binding protein or a transcription factor, or homologous to a DNA binding protein or a transcription factor. Most preferably, candidate nucleic acid binding molecules comprise polypeptides which are at least 40% homologous, more preferably at least 60% homologous, even more preferably at least 75% homologous or even more, for example 85%, or 90%, or even more than 95% homologous to one or more DNA binding proteins, preferably transcription factors, using one of the homology calculation algorithms defined below.

Candidate nucleic acid binding molecules may comprise, among other things, DNA binding park(s) of any protein(s), for example zinc finger transcription factors. Zif268. ATF family transcription factors, ATF1, ATF2, bZIP proteins, CHOP, NF-εB, TATA binding protein (TBP), MDM, cjun, elk, serum response factor (SRF), ternary complex factor (TCF); KRÜPPEL. Odd Skipped, even skipped and other D. melanogaster transcription factors; yeast transcription factors such as GCN4, the GAL family of galactose-inducible transcription factors: bacterial transcription factors or repressors such as lacI^{q}, or fragments or derivatives thereof. Derivatives would be considered by a person skilled in the art to be functionally and/or structurally related to the molecule(s) from which they are derived, for example through sequence homology of at least 40%.

The candidate polypeptide binding molecules or nucleic acid binding molecules may be non-randomised polypeptides, for example ‘wild-type’ or allelic variants of naturally occurring polypeptides, or may be specific mutant(s), or may be wholly or partially randomised polypeptides, preferably structurally related to nucleic acid binding proteins as described herein.

In a highly preferred embodiment, the polypeptide molecules are displayed on the surface of bacteriophage particles. Such displayed nucleic acid and polypeptide binding molecules are preferably partially randomised zinc-finger type transcription factors, preferably retaining at least 40% homology (as described herein) to zinc-finger type transcription factors.

In some cases, sequence homology may be considered in relation to structurally important residues, or those residues which are known or suspected of beings evolutionarily conserved. In such instances, residues known to be variable or non-essential for a particular structural conformation may be discounted from the homology calculation. For example, as explained herein, zinc fingers are known to have certain residues which are important for the formation of the three-dimensional zinc finger structure. In these cases. homology may be considered over about seven of said important amino acid residues amongst approximately thirty residues which may comprise the whole finger structure.

As used herein, the term homology may refer to structural homology. Structural homology may be estimated by comparing the structural RMS deviation of the main part of the carbon atom backbone of two or more molecules. Preferably, the molecules may be considered structurally homologous if the deviation is 5 Å or less, preferably 3 Å or less, more preferably 1.5 Å or less. Structurally homologous molecules will not necessarily show significant sequence homology.

Candidate nucleic acid binding molecules or polypeptide binding molecules, as defined above, may be pre-screened prior to being tested in the methods of the invention using routine assays known in art for determining the binding of molecules to nucleic acids or polypeptides so as to eliminate molecules that do not have binding ability. For example, a candidate nucleic acid binding molecule, preferably a library of candidate nucleic acid binding molecules, are contacted with nucleic acid and binding determined. The nucleic acids may for example be labelled with a detectable label, such as a fluorophore/flurochrome, such that after a wash step binding can be determined easily, for example by monitoring fluorescence. Similar methods may be used to pre-screen polypeptide binding molecules. Other methods for measuring binding to nucleic acid are set out below.

The nucleic acid with which the candidate nucleic acid binding molecules are contacted may be non-specific nucleic acids, such as a random oligonucleotide library or sonicated genomic DNA and the like. Alternatively, a specific sequence such as a specific DNA sequence or a partially randomised library of sequences may be used.

Preferably, the nucleic acid binding molecules of the invention may bind the target nucleic acid with different affinity in the presence or in the absence of a ligand. Similarly, the polypeptide binding molecules may bind their targets (i.e., where the first and second molecules are both polypeptides) with a different affinity in the presence or in the absence of ligand. The binding may be enhanced by the presence of the ligand (i.e. bind with a higher affinity in the presence of ligand), or may be reduced in the presence of ligand (i.e. bind with a lower affinity in the presence of ligand). In the case where association of the nucleic acid binding molecule(s) with the target nucleic acid (or the association of the polypeptide binding molecule(s) with their targets) is enhanced by the presence of ligand, said association may be additive with the binding of the ligand, or may be synergistic with the binding of the ligand, or may affect the binding in another way. If the binding is synergistic with the binding of the ligand, said binding may be either wholly or partly dependent on the presence of the ligand. Preferably, the characteristics of binding may be such that the nucleic acid binding molecule(s) or polypeptide binding molecule(s) nay be eluted by addition of an excess of the ligand.

Nucleic acid binding molecules and polypeptide binding molecules according to the invention are preferably polypeptide sequences, optionally encoded by nucleic acid sequences. Fragments, mutants, alleles and other derivatives of the molecules of the invention preferably retain substantial homology with said sequences). As used herein. “homology” means that the two entities share sufficient characteristics for the skilled person to determine that they are similar. Preferably, homology is used to refer to sequence identity. Thus, the derivatives of said nucleic acid binding molecules of the invention and polypeptide binding molecules of the invention, preferably retain substantial sequence identity with said molecules.

In the context of the present invention, a homologous sequence is taken to include any sequence which is at least 60, 70, 80 or 90% identical, preferably at least 95 or 98% identical over at least 5, preferably 8, 10, 15, 20, 30, 40 or even more residues or bases with the molecules (i.e., the sequences thereof) of the invention, for example as shown in the sequence listing herein. In particular, homology should typically be considered with respect to those regions of the molecule(s) which may be known to be functionally important rather than non-essential neighbouring sequences. Although homology can also be considered in terms of similarity (i.e., amino acid residues having similar chemical properties/functions), in the context of the present invention it is preferred to express homology in terms of sequence identity.

Homology comparisons can be conducted by eye, or more usually. With the aid of readily available sequence comparison programs. These commercially available computer programs can calculate % homology between two or more sequences.

% homology may be calculated over contiguous sequences, i.e., one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and 4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin. U.S.A.: Devereux et al. 1984. Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol. 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Nucleic acid binding molecules and polypeptide binding molecules according to the invention may include any atom, ion, molecule, macromolecule (for example polypeptide). or combination of such entities that are capable of binding to nucleic acids, such as DNA. or (in the case of polypeptide binding molecules) polypeptides. Advantageously, nucleic acid binding molecules according to the invention may include families of polypeptides with known or suspected nucleic acid binding motifs. These may include for example zinc finger proteins (see below). Molecules according to the invention may also include helix-turn-helix proteins, homeodomains, leucine zipper proteins, helix-loop-helix proteins or sheet motifs which are well known to a person skilled in the art.

Polypeptide binding molecules of the invention advantageously contain protein-binding motifs, such as protein dimerization motifs as known in the art. Examples of a protein-binding motifs include the tetratricopeptide repeat (TPR) which is found in proteins associated with multiprotein complexes (Blatch and Lassle, 1999, Bioessays 21, 932-9), the Arg-Gly-Asp-Ser found in multimerin (Hayward 1997, Clin Invest Med 20, 176-87), the LXCXE motif found in SV40 Large T antigen necessary for binding to p53 protein (DeCaprio 1999. Biologicals 27, 23-8), the C-terminal VXI motif of ABP, which mediates binding of ABP to GluR2/3 through a Class I PDZ interaction to form homodimers and heteromultimers (Srivastava and Ziff, Ann N Y Acad Sci 868, 561-4), as well as the conserved Ran-binding motif found in species from yeasts to mammals (Seki et al, 1996, J Biochem (Tokyo). 120. 207-14).

According to the invention, nucleic acid binding motifs such as DNA binding motifs of one or more known or suspected nucleic acid/DNA binding polypeptide(s), or polypeptide binding motifs, may advantageously be randomised, in order to provide libraries of candidate nucleic acid binding molecules.

Crystal structures may advantageously be used in selecting or predicting the relevant binding regions of nucleic acid and polypeptide binding proteins by methods known in the art. Nucleic acid binding regions of proteins within the same structural family are often conserved or homologous to one another, for example zinc finger a-helices, the leucine zipper basic region, homeodomain helix 3.

General considerations and rules governing the binding of several polypeptide families to nucleic acids are set out in the literature, e.g., in (Suzuki et al. 1994:PNAS vol 91 pp 1X257-61). Nucleic acid binding criteria for zinc fingers as preferred nucleic acid binding molecules according to the present invention are set out in this application (see above).

It is also envisaged that the methods of the present invention could be advantageously applied to the selection of ligand-modulatable nucleic acid binding molecules from other families of transcription factors, for example from the helix-turn-helix (HTH) family and/or from the probe helix (PH) family, and/or from the C4 Zinc-binding family (which includes the hormone receptor (HR) family), from the Gal4 family. from the c-myb family, from other zinc finger families, or from any other family of nucleic acid binding proteins or polypeptide binding proteins known to one skilled in the art.

One or more polypeptides from one or more of these families could be advantageously randomised to provide a library of candidate molecules for use in the methods of the invention. Preferably, the amino acid residues known to be important for nucleic acid or polypeptide binding could be randomised. However, it may be desirable to randomise other regions of the binding molecule since alterations to the amino acid sequence outside of those elements of secondary structure that present amino acids that contact the nucleic acid or polypeptide are likely to cause conformational changes that may affect the binding properties of the molecule.

For example, randomisation may involve alteration of zinc finger polypeptides, said alteration being accomplished at the DNA or protein level. Mutagenesis and screening of zinc finger polypeptides may be achieved by any suitable means. Preferably, the mutagenesis is performed at the nucleic acid level, for example by synthesising novel genes encoding mutant polypeptides and expressing these to obtain a variety of different proteins. Alternatively, existing genes can themselves be mutated, such as by site-directed or random mutagenesis, in order to obtain the desired mutant genes.

Mutations may be performed by any method known to those of skill in the art. Preferred, however, is site-directed mutagenesis of a nucleic acid sequence encoding the protein of interest. A number of methods for site-directed mutagenesis are known in the art, from methods employing single-stranded phage such as M13 to PCR-based techniques (see “PCR Protocols: A guide to methods and applications”, M. A. Innis. D. H. Gelfand, J. J. Sninsky, T. J. White (eds.). Academic Press, New York, 1990). Preferably, the commercially available Altered Site II Mutagenesis System (Promega) may be employed, according to the manufacturer's instructions.

Randomisation of the zinc finger binding motifs is preferably directed to those amino acid residues where the code provided herein gives a choice of residues (see below). For example, positions +1, +5 and +8 are advantageously randomised, whilst preferably avoiding hydrophobic amino acids; positions involved in binding to the nucleic acid, notably −1, +2, +3 and +6, may be randomised also, preferably within the choices provided by the rules of the present invention.

Screening of the proteins produced by mutant genes is preferably performed by expressing the genes and assaying the binding ability of the protein product. A simple and advantageously rapid method by which this may be accomplished is by phage display, in which the mutant polypeptides are expressed as fusion proteins with the coat proteins of filamentous bacteriophage, such as the minor coat protein pII of bacteriophage m13 or gene III of bacteriophage Fd, and displayed on the capsid of bacteriophage transformed with the mutant genes. The target nucleic acid sequence or target polypeptide is used as a probe to bind directly to the protein on the phage surface and select the phage possessing advantageous mutants, by affinity purification. The phage are then amplified by passage through a bacterial host, and subjected to further rounds of selection and amplification in order to enrich the mutant pool for the desired phage and eventually isolate the preferred clone(s). Detailed methodology for phage display is known in the art and set forth, for example, in U.S. Pat. No. 5,123,409: Choo and Klug, (1995) Current Opinion in Biotechnology 6:431436; Smith, (1985) Science 228:1315-1317; and McCafferty et al. (1990) Nature 348:552-554; all incorporated herein by reference. Vector systems and kits for phage display are available commercially, for example from Pharmacia.

Specific peptide ligands such as zinc finger polypeptides may moreover be selected for binding to targets by affinity selection using large libraries of peptides linked to the C-terminus of the lac repressor Laci (Cull et al., (1992) Proc Natl Acad Sci USA, 89, 1865-9). When expressed in E. coli the repressor protein physically links the ligand to the encoding plasmid by binding to a lac operator sequence on the plasmid.

An entirely in vitro polysome display system has also been reported (Mattheakis et al. (1994) Proc Natl Acad Sci U S A. 91, 9022-6) in which nascent peptides are physically attached via the ribosome to the RNA which encodes them. Furthermore, polypeptides may be partitioned in physical compartments for example wells of an in vitro dish, or subcellular compartments, or in small fluid particles or droplets such as emulsions; further teachings on this topic may be found in Griffith et al., (see WO 99/02671).

A library for use in the invention may be randomised at those positions for which choices are given as set out below. The rules are intended allow the person of ordinary skill in the art to make informed choices concerning the desired codon usage at the given positions.

The recognition helix of PH family polypeptides contains conserved Arg/Lys residues which are important structural elements involved in the binding of phosphates in the nucleic acid. Base specificity is attributed to amino acids 1, 4, 5 and 8 of the helix.

These residues could be advantageously varied, for example amino acid I could be selected from Asn, Asp, His, Val, lie to provide the possibility of binding to A, C, G, or T. Similarly, amino acid 4 could be selected from Asn, Asp, His, Val, Ile, Gln, Glu, Arg, Lys. Met, or Leu to provide the possibility of binding to A, C, G or T. Preferably, the rules laid out in (Suzuki et al. 1994: PNAS vol 91 pp 12357-61) would be used in order to randomise those amino acids which affect interaction of the molecule with the nucleic acid. whether in a base specific manner, or via binding to the phosphate backbone, thereby producing a library of candidate nucleic acid binding molecules for use in the methods of the invention.

Similarly, polypeptide molecules of the helix-turn-helix family could be randomised to produce a library of candidate molecules, at least some of which may preferably be capable of binding nucleic acid in a ligand-dependent manner when used in the methods of the present invention. In particular, amino acids 1, 2, 5 and 6 are known to be conserved and function in base-specific nucleic acid binding in HTH motifs. Therefore, at least amino acids 1, 2, 5 or 6 would preferably be randomised so as to produce molecules for use according to the present invention. More preferably, amino acids 1, 5 and 6 could be selected from Asn, Asp, His, Val, Ile, Glu, Gln, Arg, Met, Lys or Leu, and amino acid 2 could be selected from Asn, Asp, His, Val, Ile, Glu, Gln, Arg, Met, Lys, Leu. Cys, Ser, Thr, or Ala.

Another family of transcription factors which may be advantageously employed in the methods of the current invention are the C4 family which includes hormone receptor type transcription factors. It is envisaged that polypeptides of this family could advantageously be used to provide candidate molecules for use in selecting nucleic acid binding molecules whose association with nucleic acid is modulatable by a nucleic acid binding ligand. Amino acids 1, 4, 5 and 9 of the C4 motif are known to be involved in contacting the DNA, and therefore these residues would preferably be altered to provide a 30 plurality of different molecules which may bind DNA in a ligand dependent manner.

Preferably, amino acids 1 and 5 could be selected from Asn, Asp, His, Val, Ile, Glu, Gln, Arg, Met, Lys or Leu, and amino acids 4 and 9 could be selected from Gln, Glu, Arg, Lys, Leu or Met.

Particularly preferred examples of DNA binding molecules are Cys)-His2 zinc finger binding proteins which, as is well known in the art, bind to target nucleic acid sequences via a-helical zinc metal atom coordinated binding motifs known as zinc fingers. Each zinc finger in a zinc finger nucleic acid binding protein is responsible for determining binding to a nucleic acid triplet, or an overlapping quadruplet, in a nucleic acid binding sequence. Preferably, there are 2 or more zinc fingers, for example 2, 3, 4, 5 or 6 zinc fingers, in each binding protein. Advantageously, there are 3 zinc fingers in each zinc finger binding protein.

Thus, in one embodiment, the invention provides a method for preparing a DNA binding polypeptide of the Cys2-His2 zinc finger class capable of binding to a target DNA G15 ) sequence, wherein binding is via a zinc finger DNA binding motif of the polypeptide, and wherein said binding is modulatable by a ligand.

All of the DNA binding residue positions of zinc fingers, as referred to herein, are numbered from the first residue in the a-helix of the finger, ranging from +1 to +9. “−1” refers to the residue in the framework structure immediately preceding the x-helix in a Cys2-His2 zinc finger polypeptide. Residues referred to as “++” are residues present in an adjacent (C-terminal) finger. Where there is no C-terminal adjacent finger. “++”interactions do not operate.

The present invention is in one aspect concerned with the production of what are essentially artificial nucleic acid binding proteins such as DNA binding proteins as well as polypeptide binding molecules such as proteins. In these proteins, artificial analogues of amino acids may be used, to impart the proteins with desired properties or for other reasons. Thus, the term “amino acid”, particularly in the context where “any amino acid” is referred to, means any sort of natural or artificial amino acid or amino acid analogue that may be employed in protein construction according to methods known in the art. Moreover, any specific amino acid referred to herein may be replaced by a functional analogue thereof, particularly an artificial functional analogue. The nomenclature used herein therefore specifically comprises within its scope functional analogues or rimetics of the defined amino acids.

The α-helix of a zinc finger binding protein aligns antiparallel to the nucleic acid strand, such that the primary nucleic acid sequence is arranged 3′ to 5′ in order to correspond with the N terminal to C-terminal sequence of the zinc finger. Since nucleic acid sequences are conventionally written 5′ to 3,′, and amino acid sequences N-terminus to C-terminus, the result is that when a nucleic acid sequence and a zinc finger protein are aligned according to convention, the primary interaction of the zinc finger is with the − strand of the nucleic acid, since it is this strand which is aligned 3′ to 5′. These conventions are followed in the nomenclature used herein. It should be noted, however. that in nature certain fingers, such as finger 4 of the protein GL1, bind to the + strand of nucleic acid: see Suzuki et al., (1994) NAR 22:3397-3405 and Pavletich and Pabo, (1993) Science 261:1701 -1707. The incorporation of such fingers into DNA binding molecules according to the invention is envisaged.

The present invention may be integrated with the rules set forth for zinc finger polypeptide design in our copending European or PCT patent applications having publication numbers: WO 98/53057, WO 98/53060, WO 98/53058, WO 98/53059. describe improved techniques for designing zinc finger polypeptides capable of binding desired nucleic acid sequences. In combination with selection procedures, such as phage display, set forth for example in WO 96/06166, these techniques enable the production of zinc finger polypeptides capable of recognising practically any desired sequence.

In a preferred aspect, therefore, the invention provides a method for preparing a DNA binding polypeptide of the Cys2-His2 zinc finger class capable of binding to a target DNA sequence, wherein said binding is modulatable by a ligand, and wherein binding to each base of the triplet by an a-helical zinc finger DNA binding motif in the polypeptide is determined as follows:

a) if the 5′ base in the triplet is G, then position +6 in the cc-helix is Arg and/or position is Asp;

b) if the 5 base in the triplet is A, then position +6 in the α-helix is Gin or Glu and ++2 is not Asp;

c) if the 5 base in the triplet is T, then position +6 in the α-helix is Ser or Thr and position ++2 is Asp; or position +6 is a hydrophobic amino acid other than Ala;

d) if the 5 base in the triplet is C, then position +6 in the α-helix may be any amino acid. provided that position ++2 in the ax-helix is not Asp;

e) if the central base in the triplet is G, then position +3 in the α-helix is His:

f) if the central base in the triplet is A, then position +3 in the α-helix is Asn:

g) if the central base in the triplet is T, then position +3 in the α-helix is Ala, Ser, Ile, Leu. Thr or Val; provided that if it is Ala, then one of the residues at −1 or −6 is a small residue;

h) if the central base in the triplet is 5-meC, then position +3 in the α-helix is Ala, Ser, Ile, Leu, Thr or Val; provided that if it is Ala, then one of the residues at −1 or +6 is a small residue;

i) if the base in the triplet is G, then position −1 in the α-helix is Mg;

j) if the 3′ base in the triplet is A, then position −1 in the α-helix is Gin and position +2 is Ala:

k) if the 3 base in the triplet is T, then position −1 in the α-helix is Asn: or position −1 is Gin and position -2 is Ser:

l) if the 3 base in the triplet is C, then position −1 in the α-helix is Asp and Position +1 is Arg; where the central residue of a target triplet is C, the use of Asp at position +3 of a zinc finger polypeptide allows preferential binding to C over 5-meC.

The foregoing represents a set of rules which permits the design of a zinc finger binding protein specific for any given target DNA sequence.

A zinc finger binding motif is a structure well known to those in the art and defined in, for example. Miller et al., (1985) EMBO J. 4:1609-1614; Berg (1988) PNAS (USA) 85:99-102: Lee et al. (1989) Science 245:635-637; see International patent applications WO 96/06166 and WO 96/32475, corresponding to U.S. Ser. No. 08/422.107. incorporated herein by reference.

In general, a preferred zinc finger framework has the structure:

(A) X₀₋₂ C X₁₋₅ C X₉₋₁₄ H X₃₋₆^{H}/_{C}

where X is any amino acid, and the numbers in subscript indicate the possible numbers of residues represented by X.

In a preferred aspect of the present invention, zinc finger nucleic acid binding motifs may be represented as motifs having the following primary structure:

(B) X^{a} C X₂₋₄C X₂₋₃ FX^{c}XXXXLXXHXX^{b}X H-linker −1 1 2 3 4 5 6 7 8 9

wherein X (including X^{a}, X^{b} and X′) is any amino acid. X2₄ and X₁₃ refer to the presence of 2 or 4. or 2 or 3. amino acids, respectively. The Cys and His residues, which together co-ordinate the zinc metal atom, are marked in bold text and are usually invariant. as is the Leu residue at position +4 in the α-helix.

Modifications to this representation may occur or be effected without necessarily abolishing zinc finger function, by insertion, mutation or deletion of amino acids. For example it is known that the second His residue may be replaced by Cys (Krizek et al., (1991) J. Am. Chem. Soc. 13:45184523) and that Leu at +4 can in some circumstances be replaced with Arg. The Phe residue before X_{c} may be replaced by any aromatic other than Trp. Moreover, experiments have shown that departure from the preferred structure and residue assignments for the zinc finger are tolerated and may even prove beneficial in binding to certain nucleic acid sequences. Even taking this into account, however, the general structure involving an ac-helix co-ordinated by a zinc atom which contacts four Cys or His residues, does not alter. As used herein, structures (A) and (B) above are taken as an exemplary structure representing all zinc finger structures of the Cys2-His2 type.

Preferably, X^{a} is ^{F}/_{Y}-X or P-^{F}/_{Y}-X. In this context. X is any amino acid. Preferably, in this context X is E, K, T or S. Less preferred but also envisaged are Q, V, A and P. The remaining amino acids remain possible.

Preferably, X₂₄ consists of two amino acids rather than four. The first of these amino acids may be any amino acid, but S, E, K, T, P and R are preferred.

Advantageously, it is P or R. The second of these amino acids is preferably E, although any amino acid may be used.

Preferably, X^{b} is T or I. Preferably, X^{c} is S or T.

Preferably, X₂₋₃ is G-K-A. G-K-C, G-K-S or G-K-G. However, departures from the preferred residues are possible, for example in the form of M-R-N or M-R.

Preferably, the linker is T-G-E-K or T-G-E-K-P.

As set out above, the major binding interactions occur with amino acids −1 +3 and +6. Amino acids +4 and +7 are largely invariant. The remaining amino acids may be essentially any amino acids. Preferably, position +9 is occupied by Arg or Lys.

Advantageously positions +1, +5 and +8 are not hydrophobic amino acids, that is to say are not Phe, Trp or Tyr. Preferably, position ++2 is any amino acid, and preferably serine, save where its nature is dictated by its role as a ++2 amino acid for an N-terminal zinc finger in the same nucleic acid binding molecule.

In a most preferred aspect, therefore, bringing together the above, the invention allows the definition of every residue in a zinc finger DNA binding motif which will bind specifically to a given target DNA triplet.

The code provided by the present invention is not entirely rigid; certain choices are provided. For example, positions +1, +5 and +8 may have any amino acid allocation, whilst other positions may have certain options: for example, the present rules provide that, for binding to a central T residue, any one of Ala, Ser or Val may be used at 3 . In its broadest sense, therefore, the present invention provides a very large number of proteins which are capable of binding to every defined target DNA triplet.

Preferably, however, the number of possibilities may be significantly reduced. For example, the non-critical residues +1+5 and +8 may be occupied by the residues Lys, Thr and Gin respectively as a default option. In the case of the other choices, for example, the first-given option may be employed as a default. Thus, the code according to the present invention allows the design of a single, defined polypeptide (a “default” polypeptide) which will bind to its target triplet.

We also describe a method for preparing a DNA binding protein of the Cys2-His2 zinc finger class capable of binding to a target DNA sequence in a manner modulatable by a ligand, comprising the steps of: (a) selecting a model zinc finger domain from the group consisting of naturally occurring zinc fingers and consensus zinc fingers: and (b) mutating at least one of positions −1, +3, +6 (and ++2) of the finger as required by a method according to the present invention.

In general, naturally occurring zinc fingers may be selected from those fingers for which the DNA binding specificity is known. For example, these may be the fingers for which a crystal structure has been resolved: namely Zif 268 (Elrod-Erickson et a!., (1996) Structure 4:1171-1180), GLI (Pavletich and Pabo, (1993) Science 261:1701-1707), Tramtrack (Fairall et al., (1993) Nature 366:483 487) and YYI (Houbaviv et al., (1996) PNAS (USA) 93:13S77-13582).

Although mutation of the DNA-contacting amino acids of the DNA binding domain allows selection of polypeptides which bind to desired target nucleic acids, and whose binding may be modulatable by a linker which operates at the polypeptide-DNA interface, in a preferred embodiment residues which are outside the DNA-contacting region may be mutated. Mutations in such residues may affect the interaction between zinc fingers in a zinc finger polypeptide, and thus alter binding site specificity. Moreover, ligands which bind to a zinc finger polypeptide so as to influence zinc finger interaction and thus binding may be identified. Mutation of residues which affect the interaction between zinc fingers allows for selection of fingers which are modulatable by ligand binding at these sites.

The naturally occurring zinc finger 2 in Zif 268 makes an excellent starting point from which to engineer a zinc finger and is preferred.

Consensus zinc finger structures may be prepared by comparing the sequences of known zinc fingers, irrespective of whether their binding domain is known. Preferably, the consensus structure is selected from the group consisting of the consensus structure P Y K C P E C G K S FS Q K S D L V K H Q R T H T G , and the consensus structure P Y K C S E C G K A F S Q K S N L T R H Q R I H T G E K P.

The consensuses are derived from the consensus provided by Krizek et al., (1991) J. Am. Chem. Soc. 113: 45184523 and from Jacobs, (1993) PhD thesis. University of Cambridge. UK. In both cases, the linker sequences described above for joining two zinc finger motifs together, namely TGEK or TGEKP can be formed on the ends of the consensus. Thus, a P may be removed where necessary, or, in the case of the consensus terminating T G, E K (P) can be added.

When the nucleic acid specificity of the model finger selected is known, the mutation of the finger in order to modify its specificity to bind to the target DNA may be directed to residues known to affect binding to bases at which the natural and desired targets differ. Otherwise, mutation of the model fingers should be concentrated upon residues −1, +3, +6 and ++2 as provided for in the foregoing rules.

In order to produce a binding protein having improved binding, moreover, the rules provided by the present invention may be supplemented by physical or virtual modelling of the protein/DNA interface in order to assist in residue selection.

We describe a method for producing a zinc finger polypeptide capable of binding to a target DNA sequence, wherein said binding is modulatable by a ligand, comprising: (a) providing a nucleic acid library encoding a repertoire of zinc finger polypeptides, the nucleic acid members of the library being at least partially randomised at one or more of the positions encoding residues −1, 2, 3 and 6 of the α-helix of the zinc finger polypeptides: (b) displaying the library in a selection system and screening it against a target DNA sequence: (c) isolating the nucleic acid members of the library encoding zinc finger polypeptides capable of binding to the target sequence in the presence/absence of ligand: (d) selecting those members of the library isolated in (c) which bind the target nucleic acid sequence with different affinities in the presence and absence of the ligand.

Methods for the production of libraries encoding randomised polypeptides are known in the art and may be applied in the present invention. Randomisation may be total. or partial; in the case of partial randomisation, the selected codons preferably encode options for amino acids as set forth in the rules above.

Zinc finger polypeptides may be designed which specifically bind to nucleic acids incorporating the base U, in preference to the equivalent base T.

We describe a method for producing a zinc finger polypeptide capable of binding to a target DNA sequence, wherein said binding is modulatable by a ligand, comprising: (a) providing a nucleic acid library encoding a repertoire of zinc finger polypeptides each possessing more than one zinc fingers, the nucleic acid members of the library being at least partially randomised at one or more of the positions encoding residues −1, 2, 3 and 6 of the α-helix in a first zinc finger and at one or more of the positions encoding residues −1 2, 3 and 6 of the α-helix in a further zinc finger of the zinc finger polypeptides; (b) displaying the library in a selection system and screening it against a target DNA sequence; (c) assessing the affinity of the DNA binding molecules for the target DNA in the presence and absence of the ligand, and (d) isolating the nucleic acid members of the library encoding zinc finger polypeptides capable of binding to the target sequence with different affinities in the presence and absence of ligand.

In this aspect, the invention encompasses library technology described in our copending International patent application WO 98/53057, incorporated herein by reference in its entirety. WO 98/53057 describes the production of zinc finger polypeptide libraries in which each individual zinc finger polypeptide comprises more than one, for example two or three, zinc fingers: and wherein within each polypeptide partial randomisation occurs in at least two zinc fingers.

This allows for the selection of the “overlap” specificity, wherein, within each triplet, the choice of residue for binding to the third nucleotide (read 3′ to 5′ on the +strand) is influenced by the residue present at position +2 on the subsequent zinc finger. which displays cross-strand specificity in binding. The selection of zinc finger polypeptides incorporating cross-strand specificity of adjacent zinc fingers enables the selection of nucleic acid binding proteins more quickly, and/or with a higher degree of specificity than is otherwise possible.

Zinc finger binding motifs designed according to the invention may be combined into nucleic acid binding polypeptide molecules having a multiplicity of zinc fingers. Preferably, the proteins have at least two zinc fingers. In nature, zinc finger binding proteins commonly have at least three zinc fingers, although two-zinc finger proteins such as Tramtrack are known. The presence of at least three zinc fingers is preferred. Nucleic acid binding proteins may be constructed by joining the required fingers end to end, N-terminus to C-terminus. Preferably, this is effected by joining together the relevant nucleic acid sequences which encode the zinc fingers to produce a composite nucleic acid coding sequence encoding the entire binding protein.

We describe a method for producing a DNA binding protein as defined above, wherein the DNA binding protein is constructed by recombinant DNA technology, the method comprising the steps of: (a) preparing a nucleic acid coding sequence encoding two or more zinc finger binding motifs as defined above, placed N-terminus to C-terminus; (b) inserting the nucleic acid sequence into a suitable expression vector; and (c) expressing the nucleic acid sequence in a host organism in order to obtain the DNA binding protein.

A “leader” peptide may be added to the N-terminal finger. Preferably, the leader peptide is MAEEKP.

The invention also provides zinc finger polypeptides which comprise more than three zinc fingers, such as four, five, six, seven, eight or nine zinc fingers. For example. the invention comprises a zinc finger polypeptide which includes the natural zinc fingers TFIIIa and Zift68. A zinc finger protein has been engineered that contains a novel linker sequence. In the present case, the novel linker joins two three zinc finger domains, but may be used to join multiple groups of zinc finger domains or other domains used in engineered transcription factors. This linker differs from previously described linkers in that it is structured and comprises of a single non-DNA-binding zinc finger. We have found that structured linkers are more suitable for connecting zinc finger domains that bind to subsites separated by longer gaps of DNA sequence than linkers previously described, for example. the 8 and 11 amino acid linkers used to span i and 2 base pairs respectively. No linkers have been designed for spanning longer regions. The ability of structured linkers to span longer gaps is probably due to the fact that these linkers confine the relative freedom of the myo domains, thus minimising the conformational search that preceeds binding and also the entropy loss on binding.

The multiple zinc finger protein that we have engineered here is composed of zinc fingers 1-3 of TFIIIA and the three zinc fingers from Zip68 joined by zinc finger 4, including flanking sequences, of TFIIIA. We have called the zinc finger protein TFIIIAZif. Zinc finger 4 of TFIIIA does not bind DNA but acts as a linker in between the two sets of zinc fingers that are involved in DNA recognition. Despite the fact that this zinc finger does not make any base contacts within the major groove of the DNA, it is folded in the classical way, for Cys2His2 zinc fingers, around a Zn(II) ion and is folded to contain an alpha helix within its structure (Nolte et al. 1998). Although this particular finger was used 5 in this example, solely because it was a familiar structured polypetide, we believe that other tertiary structures would also be suitable for use as structured linkers.

The DNA binding site for the TFIIIAZif protein contains the DNA recognition sites for zinc fingers 1-3 of TFIIIA and the three zinc fingers of Zif 268. These are the DNA sequences GGATGGGAGAC and GCGTGGGCGT, respectively, as shown in Sequence ID 3. The six base pair sequence GTACCT in Sequence ID 3 is a spacer region of DNA that separates the two binding sites and the nucleotide composition of the DNA spacer appears to have no effect on binding of the protein. Therefore, this or other structured linkers could be used with other DNA spacers of different length and sequence.

The amino Acid Sequence of Zinc Finger 4 of TFIIIA, including the flanking sequences as used in the composite protein of the invention, is NIKICVYVCHFENCGKAFKKHNQLK VHQFSHTQQLP.

The nucleotide Sequence of Zinc Finger 4 of TFIIIA, including the flanking sequences, is

AACATCAAGATCTGCGTCTATGTGTGCCATTTTGAGAACTGTGGCAAAG
CATTCAAGAAACACAATCAATTAAAGGTTCATCAGTTCAGTCACACACAG
CAGCTGCCG.

B. Nucleic Acid Vectors Encoding Polypeptides and/or Nucleic Acid Binding Proteins

A nucleic acid encoding, a polypeptide, including a nucleic acid binding protein (which may be a DNA binding protein) as well as a polypeptide binding protein according to the invention can be incorporated into vectors for further manipulation. As used herein. vector (or plasmid) refers to discrete elements that are used to introduce heterologous nucleic acid into cells for either expression or replication thereof. Selection and use of such vehicles are .veil within the skill of the person of ordinary skill in the art. Many vectors are available, and selection of appropriate vector will depend on the intended use of the vector, i.e. whether it is to be used for DNA amplification or for nucleic acid expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR392 is suitable for most Gram-negative bacteria, the 29 plasmid origin is suitable for yeast, and various viral origins (e.g. SV40. polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e., they are capable of replication in at least one class of organisms but can be transfected into another class of organisms for expression. For example, a vector is cloned in E. coli and then the same vector is transfected into yeast, mammalian or plant cells even though it is not capable of replicating independently of the host cell chromosome. DNA may also be replicated by insertion into the host genome. However, the recovery of genomic DNA encoding the nucleic acid or polypeptide binding protein is more complex than that of episomally replicated vector because restriction enzyme digestion is required to excise nucleic acid binding protein DNA. DNA can be amplified by PCR and be directly transfected into the host cells without any replication component.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g., ampicillin, neomycin, methotrexate or tetracycline, complement auxotrophic deficiencies, or supply critical nutrients not available from complex media.

Selectable markers which may be used in fungal cells, for example yeast cells. include wild-type genes which complement auxotrophic defects in for example the Uracil (e.g. URA3 gene). Lysine (e.g. LYS2 gene), Adenine (e.g. ADE2 gene). Methionine (e.g. MET3 gene). Histidine (e.g. H15S3 gene). Tryptophan (e.g. TRP1 gene), Leucine (e.g. LEU2 gene) or other metabolic pathways. In addition, counter-selection methods are well known in the art. These enable genes to be selected against by the action of a chemical precursor which is harmless unless converted to a toxic product by the action of one or more gene(s). Examples of these include: 5-fluoro-orotic acid, which is converted to a toxic compound by the action of the URA3 gene product, α-amino-adipic acid, which is converted to a toxic compound by the LYS2 gene product; allyl alcohol, which is converted to a toxic compound by alcohol dehydrogenase activity as encoded by the ADH genes, or any other suitable selective regime known to those skilled in the art. Other selective markers are based on the expression of a gene in a fungus such as yeast which overcomes the metabolic arrest induced by, or toxicity of, a chemical entity which may be added to the growth medium or otherwise presented to the cells. Examples of these may include the KAN gene(s) which confer resistance to antibiotics such as G-148. the HIS3 gene which confers resistance to 3-amino-triazole, or the ADH2 gene which can confer resistance to heavy metal ions such as cadmium, or any other suitable genes which confer resistance to toxic or growth arresting regimes.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC 18 or pUC 19. which contain both E. coli replication origin and E. coli genetic marker conferring resistance to antibiotics, such as ampicillin.

Suitable selectable markers for mammalian cells are those that enable the identification of cells competent to take up nucleic acid binding protein or polypeptide binding protein nucleic acid, such as dihydrofolate reductase (DHFRF methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes the nucleic acid binding protein or the polypeptide binding protein. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression and cloning vectors usually contain a promoter that is recognised by the host organism and is operably linked to nucleic acid encoding nucleic acid binding protein or the nucleic acid encoding polypeptide binding protein. Such a promoter may be inducible or constitutive. The promoters are operably linked to DNA encoding the binding protein by removing the promoter from the source DNA by restriction enzyme digestion and inserting the isolated promoter sequence into the vector. Both the native nucleic acid binding protein (or polypeptide binding protein, as the case may be) promoter sequence and many heterologous promoters may be used to direct amplification and/or expression of the binding protein.

Promoters suitable for use with prokaryotic hosts include, for example, the β-lactamase and lactose promoter systems, alkaline phosphatase, the tryptophan (trp) promoter system and hybrid promoters such as the tac promoter. Their nucleotide sequences have been published, thereby enabling the skilled worker operably to ligate them to DNA encoding nucleic acid or polypeptide binding protein, using linkers or adapters to supply any required restriction sites. Promoters for use in bacterial systems will also generally contain a Shine-Delgarno sequence operably linked to the DNA encoding the nucleic acid or polypeptide binding protein.

Preferred expression vectors are bacterial expression vectors which comprise a promoter of a bacteriophage such as phagex or 17 which is capable of functioning in the bacteria. In one of the most widely used expression systems, the nucleic acid encoding the fusion protein may be transcribed from the vector by T7 RNA polymerase (Studier et al, Methods in Enzymol. 185, 60-89, 1990). In the E. coli BL21(DE3) host strain, used in conjunction with pET vectors, the T7 RNA polymerase is produced from the β-lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int-phage such as the CE6 phage which is commercially available (Novagen, Madison. USA). Other vectors include vectors containing the lambda PL promoter such as PLEX (Invitrogen, NL), vectors containing the trc promoters such as pTrcHisXpress™ (Invitrogen) or pTrc99 (Pharmacia Biotech, SE) or vectors containing the tac promoter such as pKK223-3 (Pharmacia Biotech) or PMAL (New England Biolabs. MA. USA).

Moreover, the nucleic acid binding protein or polypeptide binding protein gene according to the invention preferably includes a secretion sequence in order to facilitate secretion of the polypeptide from bacterial hosts, such that it will be produced as a soluble native peptide rather than in an inclusion body. The peptide may be recovered from the bacterial periplasmic space, or the culture medium, as appropriate.

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3-phosphate dehydrogenase (GAPDH), 3-phosphoglycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, or a promoter from the TATA binding protein (TBP) gene can be used.

Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PH00 promoter is e.g., a shortened acid phosphatase PH05 promoter devoid of the upstream regulatory elements (UAS) such as the PH05 (−173) promoter element starting at nucleotide −173 and ending at nucleotide −9 of the PH05 gene.

Binding protein gene transcription from vectors in mammalian hosts may be controlled by promoters derived from the genomes of viruses such as polyoma virus. adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g., a ribosomal protein promoter, and from the promoter normally associated with nucleic acid binding protein or polypeptide binding protein sequence, provided such promoters are compatible with the host cell systems.

Transcription of a DNA encoding nucleic acid binding protein or polypeptide binding protein by higher eukaryotes may be increased by inserting an enhancer sequence into the vector. Enhancers are relatively orientation and position independent. Many enhancer sequences are known from mammalian genes (e.g., elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to binding protein DNA, but is preferably located at a site 5′ from the promoter.

Advantageously, a eukaryotic expression vector encoding a nucleic binding protein or polypeptide binding protein according to the invention may comprise a locus control region (LCR). LCRs are capable of directing high-level integration site independent expression of transgenes integrated into host cell chromatin, which is of importance especially where the binding protein gene is to be expressed in the context of a permanently-transfected eukaryotic cell line in which chromosomal integration of the vector has occurred, or in transgenic animals.

Eukaryotic vectors may also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding nucleic acid or polypeptide binding protein.

An expression vector includes any vector capable of expressing nucleic acid binding protein nucleic acids and polypeptide binding protein nucleic acids that are operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, DNAs encoding relevant binding protein may be inserted into a vector suitable for expression of cDNAs in mammalian cells, e.g., a CMV enhancer-based vector such as pEVRF (Matthias, et al. (1989) NAR 17, 6418).

In a preferred embodiment, the nucleic acid binding protein and polypeptide binding protein constructs of the invention are expressed in plant cells under the control of transcriptional regulatory sequences that are known to function in plants. The regulatory sequences selected will depend on the required temporal and spatial expression pattern of the binding protein in the host plant. Many plant promoters have been characterised and would be suitable for use in conjunction with the invention. By way of illustration, some examples are provided below:

A large number of promoters are known in the art which direct expression in specific tissues and organs (e.g., roots, leaves, flowers) or in cell types (e.g. leaf epidermal cells, leaf mesophyll cells, root cortex cells). For example, the maize PEPC promoter from the phosphoenol carboxylase gene (Hudspeth & Grula Plant Mol. Bio. 12: 579-589 (1989)) is green tissue-specific; the trpA gene promoter is pith cell-specific (WO 93/07278 to Ciba-Geigy); the TA19 promoter is pollen-specific (Mariani et al. Nature 347: 737-741 (1990): Mariani et al. Nature 357: 384-387 (1992)).

Other promoters direct transcription under conditions of presence of light or absence or light or in a circadian manner. For example, the GS2 promoter described by Eddwards and Cortizi, Plant Cell 1: 241-248 (1989) is induced by light, whereas the AS1 promoter described by Tsai and Coruzzi, EMBO J 9: 323-332 (1990) is expressed only in conditions of darkness.

Other promoters are wound-inducible and typically direct transcription not just on wound induction, but also at the sites of pathogen infection. Examples are described by Xu et al. (Plant Mol. Biol. 22: 573-588 (1993));Logemann et al. (Plant Cell 1: 151-158 (1989)): and Firek et al. (Plant Mol Biol 22: 129-142 (1993)).

A number of constitutive promoters can be used in plants. These include the Cauliflower Mosaic Virus 35S promoter (U.S. Pat. No. 5,352,605 and U.S. Pat. No. 5,322,938. both to Monsanto) including minimal promoters (such as the −46 or −90 CaMV 35S promoter) linked to other regulatory sequences, the rice actin promoter (McElroy et al. Mol. Gen. Genet. 231: 150-160 (1991)), and the maize and sunflower ubiquitin promoters (Christensen et al. Plant Mol Biol. 12: 619-632 (1989); Binet et a!. Plant Science 79: 87-94 (1991)).

Using promoters that direct transcription in the plant species of interest, the nucleic acid or polypeptide binding protein of the invention can be expressed in the required cell or tissue types. For example, if it is the intention to utilise the nucleic acid or polypeptide binding protein to regulate a gene in a specific cell or tissue type, then the appropriate promoter can be used to direct expression of the binding protein construct.

An appropriate terminator of transcription is fused downstream of the selected binding protein containing transgene and any of a number of available terminators can be used in conjunction with the invention. Examples of transcriptional terminator sequences that are known to function in plants include the nopaline synthase terminator found in the pB1 vectors (Clontech catalog 1993/1994), the E9 terminator from the rbcS gene (rej), and the tm1 terminator from Cauliflower Mosaic Virus.

A number of sequences found within the transcriptional unit are known to enhance gene expression and these can be used within the context of the current invention. Such sequences include intron sequences which particularly in monocotyledonous cells, are known to enhance expression. Both intron 1 of the maize Adh1 gene and the intron from the maize bronze1 gene have been found to be effective in enhancing expression in maize cells (Callis et al. Genes Develop. 1: 1183-1200 (1987)) and intron sequences are frequently incorporated into plant transformation vectors, typically within the non-translated leader.

A number of virus-derived non-translated leader sequences have been found to enhance expression, especially in dicotyledonous cells. Examples include the “Ω” leader sequence of Tobacco Mosaic Virus, and similar leader sequences of Maize Chlorotic Mottle Virus and Alfalfa Mosaic Virus (Gallie et al. Nucl. Acids Res. 15: 8693-8711 (1987); Shuzeski et al. Plant Mol Biol, 15: 65-79 (1990)).

The nucleic acid binding proteins of the current invention are targeted to the cell nucleus so that they are able to interact with host cell DNA and bind to the appropriate DNA target in the nucleus and regulate transcription. It may also be desirable to target the polypeptide binding proteins of the invention to the nucleus, if this is where the target polypeptides bound by the polypeptide binding proteins are located, and/or where the activity modulated by binding of the proteins to each other is to be expressed.

To effect this, a Nuclear Localisation Sequence (NLS) is incorporated in frame with the construct, for example the expressible zinc finger construct. The NLS can We fused either 5′ or 3′ to the protein encoding sequence.

The NLS of the wild-type Simian Virus 40 Large T-Antigen (Kalderon et al. Cell 37: 801-813 (1984); Markland et al. Mol. Cell Biol. 7: 4255-4265 (1987)) is an appropriate NLS and has previously been shown to provide an effective nuclear localisation mechanism in plants (van der Krol et al. Plant Cell 3: 667-675 (1991)). However, several Alternative NLSs are known in the art and can be used instead of the SV40 NLS sequence. these include the Nuclear Localisation Signals of TGA-IA and TGA-IB (van der Krol et al.; Plant Cell 3: 667-675 (1991)).

A variety of transformation vectors are available for plant transformation and the nucleic acid or polypeptide binding protein encoding genes of the invention can be used in conjunction with any such vectors. The selection of vector will depend on the preferred transformation technique and the plant species which is to be transformed. For certain target species, different selectable markers may be preferred.

For Agrobacterium-mediated transformation, binary vectors or vectors carrying at least one T-DNA border sequence are suitable. A number of vectors are available including pBIN19 (Bevan, Nucl. Acids Res. 12: 8711-8721 (1984), the pBI series of vectors, and pCIB10 and derivatives thereof (Rothstein et al. Gene 53: 153-161 (1987); WO 95/33818 to Ciba-Geigy).

Binary vector constructs prepared for Agrobacterium transformation are introduced into an appropriate strain of Agrobacterium tumefaciens (for example, LBA 4044 or GV 3101) either by triparental mating (Bevan; Nucl. Acids Res. 12: 8711-8721 (1984)) or direct transformation (Hofgen & Willmitzer, Nucl. Acids Res. 16: 9877 (1988)).

For transformation which is not Agrobacterium-mediated (Le, direct gene transfer), any vector is suitable and linear DNA containing only the construct of interest may be preferred. Direct gene transfer can be undertaken using a single DNA species or multiple DNA species (co-transformation; Schroder et al. Biotechnology 4: 1093-1096 (1986)).

Particularly useful for practising several embodiments of the present invention are expression vectors that provide for the transient expression of DNA encoding a nucleic acid nucleic acid or polypeptide binding protein in plant cells or mammalian cells. Transient expression usually involves the use of an expression vector that is able to replicate efficiently in a host cell, such that the host cell accumulates many copies of the expression vector, and, in turn, synthesises high levels of nucleic acid or polypeptide binding protein. For the purposes of the present invention, transient expression systems are useful e.g., for identifying DNA binding protein mutants, to identify potential phosphorylation sites, or to characterise functional domains, for example domains which mediate protein-protein interaction, of the protein.

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing DNA binding protein expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis) or in situ hybridisation, using an appropriately labelled probe which may be based on a sequence provided herein. Those skilled in the art will readily envisage how these methods may be modified, if desired.

In accordance with another embodiment of the present invention, there are provided cells containing the above-described nucleic acids. Such host cells such as prokaryote. yeast and higher eukaryote cells may be used for replicating DNA and producing the DNA binding protein. Suitable prokaryotes include eubacteria, such as Gram-negative or Gram-positive organisms, such as E. coli, e.g. E. coli K-12 strains, DH5c and HB 101, or Bacilli. Further hosts suitable for the nucleic acid or polypeptide binding protein encoding vectors include eukaryotic microbes such as filamentous fungi or yeast, e.g. Saccharomyces cerevisiae. Higher eukaryotic cells include plant cells and animal cells such as insect and vertebrate cells, particularly mammalian cells including human cells, or nucleated cells from other multicellular organisms. In recent years propagation of vertebrate cells in culture (tissue culture) has become a routine procedure. Examples of useful mammalian host cell lines are epithelial or fibroblastic cell lines such as Chinese hamster ovary (CHO) cells, NIH 3T3 cells. HeLa cells or 293T cells. The host cells referred to in this disclosure comprise cells in in vitro culture as well as cells that are within a multicellular host organism.

DNA may be stably incorporated into cells or may be transiently expressed using methods known in the art. Stably transfected cells may be prepared by transfecting cells with an expression vector having a selectable marker gene, and growing the transfected cells under conditions selective for cells expressing the marker gene. To prepare transient transfectants, cells are transfected with a reporter gene to monitor transfection efficiency.

To produce such stably or transiently transfected cells, the cells should be transfected with a sufficient amount of the nucleic acid or polypeptide binding protein-encoding nucleic acid to form the relevant binding protein. The precise amounts of DNA encoding the nucleic acid or polypeptide binding protein may be empirically determined and optimised for a particular cell and assay.

Host cells are transfected or, preferably, transformed with the above-mentioned expression or cloning vectors of this invention and cultured in conventional nutrient media modified as appropriate for inducing promoters, selecting transformants, or amplifying the genes encoding the desired sequences. Heterologous DNA may be introduced into host cells by any method known in the art, such as transfection with a vector encoding a heterologous DNA by the calcium phosphate coprecipitation technique or by electroporation. Numerous methods of transfection are known to the skilled worker in the field. Successful transfection is generally recognised when any indication of the operation of this vector occurs in the host cell. Transformation is achieved using standard techniques appropriate to the particular host cells used.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g. Sambrook et al. (1989) Molecular Cloning: A Laboratory Manual, Second Edition, Cold Spring Harbor Laboratory Press).

Transfected or transformed cells are cultured using media and culturing methods known in the art, preferably under conditions whereby the nucleic acid or polypeptide binding protein encoded by the DNA is expressed. The composition of suitable media is known to those in the art so that they can be readily prepared. Suitable culturing media are also commercially available.

Transformation of plant cells is normally undertaken with a selectable marker which may provide resistance to an antibiotic or to a herbicide. Selectable markers that are routinely used in transformation include the nptII gene which confers resistance to kanamycin (Messing & Vierra Gene 19: 259-268 (1982); Bevan et al. Nature 304: 184-187 (1983)), the bar Lene which confers resistance to the herbicide phosphinothricin (White et al. Nucl. Acids Res. 18: 1062 (1990); Spencer et al. Theor. Appl. Genet. 79: 625-631 1990)), the hph gene which confers resistance to the antibiotic hygromycin (Blochlinger & Diggelmann Mol. Cell Biol. 4: 2929-2931 (1984)), and the dhfr gene which confers resistance to methotrexate (Bourouis et al. EMBO J 2: 1099-1104 (1983)). More recently. a number of selection systems have been developed which do not rely of selection for resistance to antibiotic or herbicide. These include the inducible isopentyl transferase system described by Kunkel et al. (Nature Biotechnology 17: 916-919 (1999).

Although specific protocols may vary from species to species, transformation techniques are well knows in the art for most commercial plant species.

In the case of dicotyledonous species, Agrobacterium-mediated transformation is generally a preferred technique as it has broad application to many dicotyledonous species and is generally very efficient. Agrobacterium-mediated transformation generally involves the co-cultivation of Agrobacterium with explants from the plant and follows procedures and protocols that are known in the art. Transformed tissue is generally regenerated on medium carrying the appropriate selectable marker. Protocols are known in the art for many dicotyledonous crops including (for example) cotton, tomato, canola and oilseed rape, poplar, potato, sunflower, tobacco and soybean (see for example EP 0 317 511, EP 0 249 432, WO 87/07299, U.S. Pat. No. 5,795,855).

In addition to agrobacterium-mediated transformation, various other techniques can be applied to dicotyledons. These include PEG and electroporation-mediated transformation of protoplasts, and microinjection (see for example Potrykus et al. Mol. Gen. Genet. 199: 169-177 (1985); Reich et al. Biotechnology 4: 1001-1004 (1986); Klein et al. Nature 327: 70-73 (1987)). As with Agrobacterium-mediated transformation. transformed tissue is generally regenerated on medium carrying the appropriate selectable marker using standard techniques known in the art.

Although, agrobacterium-mediated transformation has been applied successfully to monocotyledonous species such as rice and maize and protocols for these approaches are available in the art, the most widely used transformation techniques for monocotyledons remain particle bombardment, and PEG and electroporation-mediated transformation of protoplasts.

In the case of maize. Gordon-Kamm et al (Plant Cell 2: 603-618 (1990)), Fromm et al. (Biotechnology 8: 833-839 (1990) and Koziel et al. (Biotechnology 1: 194-200 (1993)) have published techniques for transformation using particle bombardment.

In the case of rice, protoplast-mediated transformation for both Japonica- and Indica-types has been described (Zhang et al. Plant Cell Rep. 7: 379-384 (1988); Shimamoto et al. Nature 3,8: 274-277: Datta et al. Biotechnology 8: 736-7 40 (1990)) and both types are also routinely transformable using particle bombardment (Christou et al. Biotechnology 9: 957-962 (1991)).

In the case of wheat, transformation by particle bombardment has been described for both type C long-term regenerable callus (Vasil et al. Biotechnology 10: 667-674 (1992)) and immature embryos and immature embryo-derived callus (Vasil et al., Biotechnology 11: 1553-1558 (1993); Weeks et al. Plant Physiol. 102: 1077-1084 (1993)). A further technique is described in published patent applications WO 94/13822 and WO 95/33818.

The nucleic acid and polypeptide binding protein constructs of the invention are suitable for expression in a variety of different organisms. However, to enhance the efficiency of expression it may be necessary to modify the nucleotide sequence encoding the nucleic acid or polypeptide binding protein to account for different frequencies of codon usage in different host organisms. Hence it is preferable that the sequences to be introduced into organisms, such as plants, conform to preferred usage of codons in the host organism.

In general, high expression in plants is best achieved from codon sequences that have a GC content of at least 35% and preferably more than 45%. This is thought to be because the existence of ATT′A motifs destabilise messenger RNAs and the existence of AATAAA motifs may cause inappropriate polvadenylation, resulting in truncation of transcription. Murray et al. (Nucl. Acids Res. 17: 477-498 (1989)) have shown that even within plants, monocotyledonous and dicotyledonous species have differing preferences for codon usage, with monocotyledonous species generally preferring GC richer sequences. Thus, in order to achieve optimal high level expression in plants, gene sequences can be altered to accommodate such preferences in codon usage in such a manner that the codons encoded by the DNA are not changed.

Plants also have a preference for certain nucleotides adjacent to the ATG encoding the initiating methionine and for most efficient translation, these nucleotides may be modified. To facilitate translation in plant cells, it is preferable to insert, immediately upstream of the ATG representing the initiating methionine of the gene to be expressed, a “plant translational initiation context sequence”. A variety of sequences can be inserted at this position. These include the sequence the sequence 5′-AAGGAGATATAACAATG-3′ (Prasher et al. Gene 111: 229-233 (1992); Chalfie et al. Science 263: 802-805 (1992)), the sequence 5′-GTCGACCATG-3′ (Clontech 1993/1994 catalog, page 210), and the sequence 5′-TAAACAATG-3′ (Joshi et al. Nucl. Acids Res. 15: 6643-6653 (1987)). For any particular plant species, a survey of natural sequences available in any databank (e.g. GenBank) can be undertaken to determine preferred “plant translational initiation context sequences” on a species-by-species basis.

Any changes that are made to the coding sequence can be made using techniques that are well known in the art and include site directed mutagenesis PCR, and synthetic gene construction. Such methods are described in published patent applications EP 0 385 962 (to Monsanto). EP 0 359 472 (to Lubrizol) and WO 93/07278 (to Ciba-Geigy). Well known protocols for transient expression in plants can be used to check the expression of modified genes before their transfer to plants by transformation.

C. Ligands

A ligand according to the invention is typically any molecule capable of binding to any of the other components of a switching system. For example, with regard to a gene switch, a ligand is typically capable of binding to DNA, the DNA-binding molecule or any other component of the gene expression machinery. A variety of DNA-binding liands are known in the art and include acridine orange, 9-Amino-6-chloro-2-methoxyacridine. actinomycin D, 7-aminoactinomycin D, echinomycin, dihydroethidium, ethidium-acridine heterodimer, ethidium bromide, propidium iodide, hexidium iodide, Hoechst 33258. Hoechst 33342. hydroxystibamidine, psoralen, Distamycin A, calicheamnicin oligosaccharides, triple helix forming oligos, PNA, pyrole-imidazole polyamides and synthetic peptides or peptide derivatives such as described by Lescinier et al. Chem. Eur. J. 4:425-433 (1998). Also included within the meaning of the term ligand and DNA binding molecules are molecules capable of binding RNA and/or other nucleic acids.

As applied to a protein switch, a ligand is any molecule capable of binding to the polypeptide binding molecule (including a polypeptide binding protein), or another protein. Protein binding ligands are known in the art, and include, for example. immunoglobulins, antibodies, ATP, cAMP, GABA, Fas ligand, CIDs (chemical inducers of dimerization), an FK506 and FK1012 (as described in Spencer et al. 1993. Science 262 1019), peptide hormone molecules, retinoic acid, acridine derivatives and other anticancer drugs as described in Finlay and Baguley (2000), Cancer Chemother Pharmacol 45. 417. etc.

Ligand mediated protein-protein association is described for example in Lin et al (1998). Blood 91. 890-897. Spencer et al (1993), Science 262, 1019, Keenan et al. (1998) Bioorganic and Medicinal Chemistry 6, 1309-1335 and Fan et al. (1999). HumanGene Therapy 10. 2273-2285.

Derivatives of ligands are also included provided that they are capable of binding to the nucleic acid and polypeptide components of the switching system as described herein.

In a preferred embodiment, a ligand according to the invention is capable of modulating the topology, locally or otherwise, of the nucleic acid or polypeptide to which it is bound. For example, a ligand according to the invention may be capable of modulating the topology of a juxtaposed nucleic acid sequence motif to which it is desired to bind a DNA binding molecule according to the invention, or the topology of a protein binding motif on a protein capable of binding to another protein.

Exemplary ligands for nucleic acid binding have shape and charge characteristics that allow them to reside along the DNA, in either the minor or major groove, intercalate or a combination of these.

Suitable ligands in addition to those known in the art may be selected by the use of nucleic acid or polypeptide binding assays. For example, a candidate ligand, preferably a plurality of candidate ligands, is contacted with target nucleic acid or polypeptides and binding determined. The targets may for example be labelled with a detectable label, such as a fluorophore/fluorochrome, such that after a wash step binding can be determined easily, for example by monitoring fluorescence. The target with which the candidate binding ligands are contacted may be non-specific, such as a random polypeptide or nucleic acid libraries or sonicated genomic DNA and the like. Alternatively, a specific sequence may be used, or a partially randomised library of sequences.

It is particularly preferred that ligands of the invention bind to polypeptides or DNA in a sequence and/or topology dependent manner so that binding can be restricted to a particular target, thus enhancing the specificity of the gene or protein switch. Specificity of binding may be determined, for example, by comparing the binding of the ligand to a target sequence with binding to a mixture of non-specific molecules.

Ligands according to the invention may bind conditionally to their targets. For example, psoralen is a ligand that can bind DNA covalently if illuminated at wavelengths of about 400 nm or less. Ligands capable of binding their targets in more than one manner may be employed in the current invention. Such ligands may bind or associate with the target via any one or more mechanism(s) such as outlined above.

In a preferred embodiment, libraries of ligands may be prepared. In particular, libraries of ligands may be immobilised to a solid phase, such as a substantially planar solid phase, including membranes and non-porous substrates such as plastic and glass. The resulting immobilised library may conveniently be used in high throughput screening procedures.

Particularly preferred ligands are those which are substantially non toxic to plants and or animal cells such that they may be administered to said cells and modulate binding of the nucleic acid or polypeptide binding molecule without having an adverse effect on the cells. Thus it may be desirable to pre-screen compounds to exclude toxic compounds.

Furthermore, given that ligands should typically be capable of being taken up by the cells of animals or plants, preferred compounds are suitable for administration to animals and plants. For example, preferred compounds are capable of being taken up via the leaves (for foliar application) or roots of plants (for application to the soil) or of permeating seeds (for use in seed treatment). It may also preferred to use compounds that can be taken up by bacteria yeast and/or fungi that can themselves be delivered to the target host organism. The compounds should also preferably be stable in the soil and/or plant for prolonged periods. In the case of animals, preferred compounds are suitable for topical or oral adminstration.

D. Target Nucleic Acid

The term ‘target nucleic acid’ refers to any DNA or other nucleic acid for use in the methods of the invention. This nucleic acid may be of known sequence, or may be of unknown sequence. This nucleic acid may be prepared artificially in a laboratory; or may be a naturally occurring nucleic acid. This nucleic acid may be in substantially pure form, or may be in a partially purified form, or may be part of an unpurified or heterogeneous sample. Preferably, the target nucleic acid is a putative promoter or other transcription regulatory region such as an enhancer. More preferably, the target nucleic acid is in substantially pure form. Even more preferably, the target nucleic acid is of known sequence. In a most preferred embodiment, the target nucleic acid is purified nucleic acid of known sequence of a promoter from a gene of interest, for example from a gene suspected of being associated with a disease state, more preferably from a gene useful in gene therapy.

Examples of target sequences of interest include sequence motifs that are bound by transcription factors, such as zinc fingers. Particular examples include the promoters of genes involved in the biosythesis and catabolism of gibberellins (Phillips et al. Plant Physiol 108: 1049-1057 (1995). MacMillin et al., Plant Physiol 113: 1369-1377 (1997), Williams et al., Plant Physiol 117: 559-563 (1998); Thomas et al., PNAS 96: 4698-4703 (1999)); the promoters of genes whose products are responsible for ripening (such as polygalacturonase and ACC oxidase: the promoters of genes involved in the biosythesis of volatile ester, which are important flavour compounds in fruits and vegetables (Dudavera et al. Plant Cell 8: 1137-1148 (1996); Dudavera et al., Plant J. 14: 297-304 (1998); Ross et al. Arch. Biochem. Biophys. 367: 9-16 (1999)); the promoters of genes involved in the biosynthesis of pharmaceutically important compounds; and the promoters of genes encoding allergens such as the peanut allergens Arah1, Arah2 and Arah3 (Rabjohn et al. J. Clin. Invest 103: 535-542).

Other plant promoters of interest are the bronze promoter (Ralston et al. Genetics 119: 185-197 (1988) and Genbank Accession No. X07937.1) that directs expression of UDPglucose flavanoid glycosyl-transferase in maize, the patatin-1 gene promoter (Jefferson et al., Plant Mo. Biol. 14: 995-1006 (1990)) that contains sequences capable of directing tuber-specific expression, and the phenylalanine ammonia lyase promoter (Bevan et al. Embo J. 8: 1899-1906 (1989)) though to be involved in responses to mechanical wounding and normal development of the xylem and flower.

Target nucleic acid may also be provided as a plurality of sequences, for example where one or more residues in the nucleic acid sequence are varied or random. Examples of a plurality of sequences are libraries of nucleic acid sequences comprising putative zinc finger binding sites. Other sequence motifs that bind the nucleic acid binding domain of a transcription factor may also be included in the plurality of sequences, typically varied or randomised at one or more positions. For example the chemically inducible promoter fragments described above may be randomised to produce a plurality of target nucleic acid sequences for use in the screening methods of the present invention.

E. Assays

With respect to gene switches, the methods of the present invention typically involve using a tripartite configuration of one or more nucleic acid binding molecules, one or more ligands and one or more target nucleic acid sequences as described above to screen for (i) nucleic acid binding molecules that bind to a target nucleic acid in a manner that is modulatable by a ligand (ii) ligands that modulate binding of a nucleic acid binding molecule to a target nucleic acid and/or (iii) a target nucleic acid that is bound modulatably by a nucleic acid binding molecule as a result of an interaction with a ligand. With regard to protein switches, the methods of the present invention typically involve using a tripartite configuration of one or more first polypeptide molecules, one or more ligands and one or more second polypeptide as described above to screen for (i) polypeptide binding molecules that bind to a (another) target polypeptide in a manner that is modulatable by a ligand and/or (ii) ligands that modulate binding of two polypeptides to each other.

In other words the methods of the invention may be used to screen for any or all of the components of the gene switch system or protein switch system of the present invention.

Typically, one or two of the components is a known constant while two or one, respectively, of the other components are screened. For example, a given nucleic acid binding molecule and target nucleic acid may be used to screen a plurality of ligands or candidate ligands. Alternatively, a plurality of nucleic acid binding molecules and of ligands may be screened against a given target nucleic acid for a gene switch, and a plurality of polypeptide binding molecules and of ligands may be screened against a given target polypeptide for a protein switch. Other combinations are also envisaged.

Each component may be one individual molecular species or a plurality of molecular species. Where a plurality of species is used, they may be substantially all known, partially randomised or fully randomised. For example, the plurality of nucleic acid binding molecules may be a randomised zinc finger library and the plurality of target nucleic acid may be a library of nucleic acid molecules randomised at one or more typically three or more contiguous, residues. Alternatively, for a protein swvitch screen, the plurality of polypeptide molecules may be a library of polypeptides randomised at one or more locations However, all three components may be screened for simultaneously. Thus. in a preferred embodiment, the invention provides a method for isolating multiple nucleic acid or polypeptide binding molecules in the presence of multiple ligands, said nucleic acid or polypeptide binding molecules being selected using multiple target nucleic acid sequences (or target polypeptides as the case may be) in a single selection (isolation) procedure.

The library of candidate nucleic acid or polypeptide binding molecules is preferably a phage display library. In the case of nucleic acid phage libraries, individual candidate molecules of the library optionally are structurally related to zinc finger transcription factors (for example see Choo and Klug, (1994) PNAS (USA) 91:11163-67. which describes aspects of such libraries and is incorporated herein by reference). This library is preferably constructed with DNA sequences of the form GCGNNNGCG (where all 64 middle triplets are represented in the mixture).

One or more ligands means at least one ligand, preferably two, three or four ligands, more preferably five, six, or seven ligands, most preferably a mixture of eight ligands, or even more. The ligands may be in any molar ratio to one another within the mixture, but will preferably be approximately equimolar with one another. The ligands may be provided in the form of a library of ligands.

In our selection method as applied to a protein switch in which the protein components are single species and the ligand is provided in the form of a ligand library, the methods of our invention as described herein allow the selection of potential ligand molecules of interest as a first step, i.e., those which form complexes with the first and second polypeptides. Thus, ligands of interest are selected which are capable of binding to one or both of the polypeptides. As a second step, the strength of binding of the polypeptides to each other are tested in the absence or presence of the ligand component of the complex to select those complexes in which the binding between the polypeptides differs in the presence or absence of the ligand component. Our selection method therefore directly selects ligands which bind to one or both of the polypeptides, without the need for any further screen to determine whether an individual ligand molecule is capable of forming a complex with the polypeptides.

The method of our invention is preferably be carried out over at least 3, 4, 5 or 6 rounds of selection, preferably about 6 rounds of selection.

Nucleic acid or polypeptide binding molecules (such as phage clones) isolated by the above methods are preferably individually assayed (for example in microtitre plates as described below) for binding to the target nucleic acid (such as a GCGNNNGCG mixture) or a target polypeptide (as the case may be) in the presence and absence of a mixture of the ligands to identify clones which are capable of ligand-modulatable binding.

Those phage clones which are capable of ligand-modulatable binding are preferably tested in the presence of a mixture of the ligands, in order to deduce the optimum target nucleic acid or polypeptide sequence, for example using different or variant target sequences, or by the binding site signature method method for nucleic acid binding proteins (see Choo and Klug, (1994) PNAS (USA) 91:11163-67).

Where candidate nucleic acid binding or polypeptide molecules are used rather than molecules known or determined to have nucleic acid or polypeptide binding properties, the method of the invention preferably features a pre-selection step to remove candidate n binding molecules which do not require ligand to bind the nucleic acid or polypeptide.

Association of the candidate nucleic acid or polypeptide binding molecule with the target nucleic acid or polypeptide may be assessed by any suitable means known to those skilled in the art. For example, the nucleic acid or polypeptide may be immobilised by biotinylation and linking to beads such as streptavidin coated beads (Dynal). In a preferred embodiment wherein the nucleic acid or polypeptide binding molecules are phage displayed polypeptides, binding of said molecules to the nucleic acid or polypeptide may be assessed by eluting those phage which bind, and infecting logarithmic phase E. coli TG1 cells. The presence of infective particles eluted from the nucleic acid indicates that association of the nucleic acid binding molecule(s) with the nucleic acid has occurred, or that association of the polypeptide binding molecule(s) with the polypeptide has occurred in the case of a protein switch. Alternatively, association of the candidate nucleic acid or polypeptide binding molecule(s) with the target nucleic acid or polypeptide may be assessed by Scintillation Proximity Assay (SPA). For example, the target nucleic acid or polypeptide could be biotinylated and immobilised to streptavidin coated SPA beads, and the candidate nucleic acid or polypeptide binding molecules may be radioactively labelled. for example with ³⁵S-methionine where the molecules are polypeptides. Association of the candidate nucleic acid or polypeptide binding molecules with the target nucleic acid or polypeptide could then be assessed by monitoring the readout of the SPA. Alternatively, the association could be monitored by fluorescent resonance energy transfer (FRET). In this case, the target nucleic acid or polypeptide could be labelled with a donor fluor, and the nucleic acid binding molecule(s) or polypeptide(s) could be labelled with a suitable acceptor fluor. Whilst the two entities are separated, no FRET would be observed, but if association (binding) took place, then there would be a change in the amount of FRET observed, this allowing assessment of the degree of associaiton.

Association of the candidate nucleic acid or polypeptide binding molecule with the target nucleic acid or polypeptide may also be assessed by bandshift assays. Bandshift assays are conducted by measuring the mobility of one or more of the components of the assay, for example the mobility of the nucleic acid or polypeptide as it is electrophoresed through a suitable gel such as a polyacrylamide or agarose gel, as is well known to those skilled in the art. In order to assess the association of the candidate nucleic acid or polypeptide binding molecule with the target nucleic acid or polypeptide, the mobility of the nucleic acid or polypeptide (as the case may be) could be measured in the presence and absence of the candidate binding molecule. If the mobility of the target nucleic acid or polypeptide is essentially the same in the presence or absence of the candidate binding molecule, then it may be inferred that the molecules do not associate, or that the association is weak. If the mobility of the nucleic acid or polypeptide is retarded in the presence of the candidate binding molecule, then it may be inferred that the candidate molecule is associating with or binding to the nucleic acid or polypeptide.

Association of the candidate nucleic acid or polypeptide binding molecule with the target nucleic acid or polypeptide may also be assessed using filter binding assays. For example, the target nucleic acid or polypeptide molecule may be immobilised on a suitable filter, such as a nitrocellulose filter. The candidate binding molecule may then be labelled. for example radioactively labelled, and contacted with the immobilised target nucleic acid or polypeptide. The binding of or association with the target nucleic acid or polypeptide may be assessed by comparing the amount of labelled candidate nucleic acid or polypeptide binding molecule which associates with the filter only to the amount of labelled candidate nucleic acid or polypeptide binding molecule which associates with the filter-immobilised target. If more labelled candidate nucleic acid or polypeptide binding molecule associates with the immobilised nucleic acid or polypeptide than with the filter only, it may be inferred that the target molecule does indeed associate with the candidate binding molecule.

Binding affinities may be estimated by any suitable means known to those skilled in the art. Binding affinities for the purposes of this invention may be absolute or may be relative. Binding affinities may be determined biochemically, or may simply be estimated by assessing the association of the candidate nucleic acid or polypeptide binding molecule with the target nucleic acid or polypeptide as described above. As used herein, the term binding affinity may refer to a simple estimation of the association of one component of the system with another.

Another suitable detection method for nucleic acid binding proteins is the use of target nucleic acid sequences linked to reporter constructs, such as bacterial luciferase or lacZ. Preferably, the reporter gene product can be measured using optical detection techniques. By way of example, a multiarray format could be used with a different candidate ligand in each position in the array (such as a microtitre plate well) and the same library of zinc finger proteins and target nucleic acid sequences at each position. The zinc finger proteins will generally be fused to a transcriptional activation domain such as the GAL4 acidic activation domain. Transcription may then be compared in the various wells and wells showing a variation in transcription compared to a control well with no ligand may be selected and the ligand further tested to identify specific target sequences/zinc finger proteins whose interaction is affected. These further tests may again be performed using an array format in which this time the ligand is kept constant and the target sequence zinc fingers varied. Phage display techniques as described above may be used to simplify the isolation of suitable zinc finger proteins. Although described in the context of zinc fingers, this method could be applied to other nucleic acid binding molecules.

Particular assays to determine if and the extent to which a ligand modulates protein-protein interactions include a fluorescence polarization assay, as described in Keenan et al (1998) Bioorganic and Medicinal Chemistry 6, 1309-1335. Other assays described in Keenan et al (supra) include assays for inducible Fas activation and for inducible transcriptional activation.

Briefly, in the inducible Fas activation assay, two fusion proteins are constructed. each comprising amino acids 175 to 304 of human Fas together with a first polypeptide or a second polypeptide respectively. Cell line clones expressing both constructs are plated in 96-well plates and treated the next day with serial dilutions of compound (ligand or candidate ligand) at typically 1 μM maximum concentration. Wells are assayed the next day for viability with for example Alamar Blue or Trypan Blue. Controls can include, for example, untransfected cells. In the transcriptional activation assay, transcription factor fusions are expressed from the tricistronic vector pCGNN-F3p65/ZIF3/Neo. An HT1080 cell line (ATCC CCL-121) cell line which contains an integrated secreted alkaline phosphatase (SEAP) target gene under control of a minimal interleukin 2 gene promoter and 12 ZFHID1 binding sites is generated as described in Rivera et al (1996) Nature Med 2. 1028. This cell line is transiently transfected with fusion protein expressing construct, with and without incubation for 18-24 hours with the ligand or candidate ligand. Cell supernatant is removed and assayed for SEAP activity using any suitable phosphatase assay, for example, the assay described in Rivera et al (supra), taking into account background SEAP activity (as measured from mock transfected HT 1080 cells).

Ligand mediated protein-protein interaction may also be assayed by way of a modified two hybrid assay. Thus, two fusion protein constructs are made, one comprising one of a pair of protein binding partners and the GAL4 binding domain, and the other comprising the other of the pair of protein binding partners and the VP 1 6 activation domain. Expression of a reporter gene, for example, beta-galactosidase, is measured in the presence and absence of the candidate ligand.

It is envisaged that the methods of the invention may be applied in vivo, for example they could be applied to the selection or isolation of nucleic acid or polypeptide binding molecules capable of associating with target nucleic acid or polypeptide in vivo inside one or more cells, in a manner analagous to the one-hybrid system.

It is envisaged that the methods of the invention may be practised in parallel. For example, multiple target nucleic acids or polypeptides could be used in a single selective step, thereby enabling multiple nucleic acid or polypeptide binding molecules to be isolated simultaneously, even in the same physical vessel. The multiple nucleic acid or polypeptide binding molecules may preferably be different from one another. The multiple nucleic acid or polypeptide binding molecules may have similar or identical binding specificities, or may preferably have different binding specificities.

The invention may be worked using multiple ligands, either separately or in combination. For example, a target nucleic acid or polypeptide sequence may be used to isolate binding molecules according to the methods essentially as disclosed above, with the modification that more than one ligand may be present. In this way, it is possible to isolate multiple nucleic acid or polypeptide binding molecules which require different ligands to bind to the same target nucleic acid or polypeptide sequence(s).

By way of example, a particular embodiment of the method of the invention is as follows:

1. Bacterial colonies containing phage libraries that express a library of zinc fingers randomised at one or more nucleic acid binding residues (see section A.) are transferred from plates to culture medium. Bacterial cultures are grown overnight at 30° C. Culture supernatant containing phages is obtained by centrifugation.

2. 10 μmol of biotinylated target nucleic acid immobilised on 50 mg streptavidin beads (Dynal) is incubated with 1 ml of the bacterial culture supernatant diluted 1:1 with PBS containing 50 μM ZnCl₂, 4% Marvel, 2% Tween for 1 hour at 20° C. on a rolling platform as a preselection step to remove phage that bind to the target nucleic acid in the absence of a ligand.

3. After this time, 0.5 ml of phage solution is transferred to a streptavidin coated tube and incubated with biotinylated nucleic acid target site in the presence of a candidate ligand and 4 μg poly [d(I−C)]. After a one hour incubation the tubes are washed times with PBS containing 50 μM ZnCl₂ and 1% Tween, and 3 times with PBS containing 50 μM ZnCl₂ to remove non-binding phage.

4. The remaining phage are eluted using 0.1 ml 0.1 M triethylamine and the solution is neutralised with an equal volume of 1 M Tris-Cl (pH 7.4).

5. Logarithmic-phase E. coli TG I cells are infected with eluted phage, and crown overnight as described above, to prepare phage supernatants for subsequent rounds of selection.

6. After 4 rounds of selection (steps 1 to 5), bacteria are plated and phage prepared from 96 colonies are screened for binding to the nucleic acid target site in the presence and absence of the ligand. Binding reactions are carried out in wells of a streptavidin-coated microtitre plate (Boehringer Mannheim) and contain 50 μl of phage solution (bacterial culture supernatant diluted 1:1 with PBS containing 50 μM ZnCl₂, 4% Marvel, 2% Tween), 0.15 μmol nucleic acid target site and 0.25 μg poly [d(I−C)]. When added, the ligand is present at a concentration of about 1 μM.

7. After a one hour incubation the wells are washed 20 times with PBS containing 50 μM ZnCl and 1% Tween (and also ligand at a concentration of 1 μM where appropriate), and 3 times with PBS containing 50 μM ZnCl₂,

8. Bound phage are detected by ELISA (carried out in the presence of the ligand at a concentration of about 1 μM where appropriate) with horseradish peroxidase-conjugated anti-M13 IgG (Pharmacia Biotech) and quantitated using SOFTMAX 2.32 (Molecular Devices).

9. Single colonies of transformants obtained after four rounds of selection as described are grown overnight in culture. Single-stranded nucleic acid is prepared from phage in the culture supernatant and sequenced using the Sequenase™ 2.0 kit (U.S. Biochemical Corp.). The amino acid sequences of the zinc finger clones are deduced

A modification of the above example may be used to select polypeptide binding proteins. Briefly, bacteria containing phage libraries expressing a library of polypeptide binding proteins randomised at one or more residues as described above in section A are screened against a biotinylated target polypeptide or protein, which has been immobilised on streptavidin coated beads, essentially as described above. Unbound phage are washed. and bound phage are eluted and used to infect E. coli cells. After several rounds of selection, each round involving the above steps, phage are prepared and screened for binding to the target polypeptide or protein in the presence and absence of the ligand. Bound phage are detected by ELISA and identified, and the corresponding colonies are amplified, and the DNA sequence of the polypeptide binding proteins are deduced.

In the above examples, only one target nucleic acid or polypeptide sequence was used. Where a library of nucleic acid or polypeptide sequences is used, the library of sequences can be screened using the ligand and selected phage expressing the zinc finger or other protein of interest to identify specific target nucleic acid or polypeptide sequences. This may conveniently be carried out with the nucleic acid or polypeptide sequences arrayed onto a solid substrate.

In the above example, the nucleic acid or polypeptide binding molecules (e g, zinc fingers ) are present on phage. However, alternative methods for displaying the nucleic acid or polpeptide binding molecules could be used. As described in section A above, an entirely in vitro polysome display system has also been reported (Mattheakis et al., (1994) Proc Natl Acad Sci U S A. 91, 9022-6) in which nascent peptides are physically attached via the ribosome to the RNA which encodes them. Using a library of RNA, ribosomes expressing the nucleic acid or polypeptide binding molecules, screening is performed in a similar manner to the phage display method except that typically after an initial preselection step to remove nucleic acid or polypeptide binding molecules that bind in the absence of the ligand only one selection step is performed and the resulting nucleic acid or polypeptide binding molecules identified by cloning the RNA from the RNA ribosome complexes and sequencing the clones obtained.

To assist in isolating and/or identifying complexes comprising a target nucleic acid, a nucleic acid binding molecule and a ligand (or in the case of protein switches, a target protein, a polypeptide binding protein and a ligand), it may be desirable to label one or more of the components with a detectable label. For example, the nucleic acid or polypeptide may be labelled with a fluorescent tag and the nucleic acid (or polypeptide) binding molecule labelled with biotin, such that an enzyme conjugate such as streptavidin-horse radish peroxidase (HRP), that catalyses an optically detectable change in a substrate (different from the fluorescent tag) can be used. If the ligand is attached to a bead, then tripartite complexes can be detected because they will both fluoresce and give HRP activity.

A further method which is useful where multiple candidate ligands are to be screened involves the use of beads to which are attached different peptide tags. Known combinatorial chemistry techniques are used to produce a library of beads whereby the peptide tag can be used to identify unambiguously the ligand attached to the same bead. Complexes comprising the ligand, a target nucleic acid and a nucleic acid binding molecule (or a ligand, a target polypeptide and a polypeptide binding protein) can be identified by the use of labelled target and binding molecules as described above. Beads comprising a tripartite complex can then be selected and the identity of the tag determined by spectroscopy techniques which will then give the identity of the ligand.

In general, a bead format is advantageous since it allows easier isolation of productive tripartite complexes and prescreening.

We describe a method by which nucleic acid binding molecules according to the invention may be advantageously used to determine the sequence composition of a sample of target nucleic acid. For example, a nucleic acid binding molecule according to the invention may be prepared which binds to a known target nucleic acid sequence. By applying this molecule to, or contacting it with, one or more test nucleic acid samples and monitoring its binding thereto, it is possible to determine whether said nucleic acid sample(s) contain the cognate nucleic acid recognition site of the nucleic acid binding molecule, and therefore derive information about the nucleotide composition of said nucleic acid test sample(s). Such analyses may be advantageously conducted using the binding site signature method (see Choo and Klug, (1994) PNAS (USA) 91:11163-67).

Individual phage clones could advantageously be assayed for binding of their cognate nucleic acid sequence(s) in the presence or absence of individual ligands, to monitor which particular ligand modulates binding, i.e., binding between the nucleic acid and the nucleic acid binding molecule, or binding between a protein and a polypeptide binding molecule such as a protein.

Clearly, it may be that more than one ligand modulates binding of nucleic acid or polypeptide binding molecules to their cognate nucleic acid or polypeptide sequence(s). Preferably, individual nucleic acid or polypeptide binding molecules (i.e., phage clones) may be assayed for binding to target sequence(s) in the presence of discrete ligand mixtures. wherein each ligand mixture preferably contains a unique mixture of ligands. In this way, the particular ligands which may modulate binding of a particular nucleic acid or polypeptide binding molecule to its cognate target sequence may advantageously be determined. For example, if it is found that two mixtures—one lacking ligand X and the other lacking ligand Y—are incapable of inducing binding, then a mixture of ligands X and Y may have the effect of modulating the binding. This could advantageously be further investigated according to the methods of the invention as described herein.

It is envisaged that this invention may be advantageously used in the isolation of a ligand that is capable of modulating the association of a particular nucleic acid binding molecule or a particular polypeptide binding molecule with its target nucleic acid or polypeptide sequence.

We describe a method for isolating one or more ligands, said ligands each binding one or more target nucleic acid or polypeptide sequence(s), wherein said binding to one or more target nucleic acid or polypeptide sequence(s) modulates the binding of one or more nucleic acid or polypeptide binding molecules respectively, and wherein said nucleic acid or polypeptide binding molecule(s) and said ligands are different, said method comprising:

(a) providing one or more target nucleic acid or polypeptide molecule(s); (b) contacting the target nucleic acid or polypeptide molecule(s) with one or more nucleic acid or polypeptide binding molecule(s), (c) providing a library of candidate ligands, (d) assessing the ability of candidate ligands to modulate the association of the nucleic acid or polypeptide binding molecule(s) with the respective target nucleic acid or polypeptide molecule(s); and (e) isolating those candidate ligands which modulate the association of the nucleic acid or polypeptide binding molecule(s) with the target nucleic acid or polypeptide molecule(s).

In order to remove nucleic acid or polypeptide binding molecules (for example phage displayed polypeptides) which bind nucleic acid or polypeptide in a ligand-independent manner from a library a pre-selection step may optionally be performed in the absence of ligand prior to each round of selection. This step removes from the library those clones which do not require ligand for nucleic acid or polypeptide binding. Optionally, candidate molecules selected in this manner may be screened by ELISA for binding to the nucleic acid or polypeptide target in the presence or absence of the ligand(s).

It is envisaged that the methods of the current invention may be advantageously applied to the selection of nucleic acid binding molecules capable of binding nucleic acids other than DNA, for example RNA. Structural considerations of RNA binding molecules are discussed in Afshar et al (Afshar et al. 1999: Curr. Op. Biotech, vol 10 pages 59-63). In particular, ligands suitable for use in the methods of the invention as applied to RNA include those ligands described above, or may be selected from aminoglycosides and their derivatives such as paromomycin, neomycin (for examples see Park et al. 1996: J. Am. Chem. Soc, vol 118 pp10150-10155); aminoglycoside mimetics (Tok and Rando 1998: J. Am. Soc. Chem, vol 120 pp 8279-8280); acridine derivatives (for examples see Hamy et al. 1998: Biochemistry vol 37 pp5086-5095); small peptides (“aptamers”); polycatomic compounds (for examples see Wang et al, 1998: Tetrahedron 54 pp7955-7976) or any other nucleic acid binding molecules known to those skilled in the art. In a preferred embodiment, derivatives or libraries of said nucleic acid binding ligands may be prepared.

Accordingly, we describe a method for isolating an RNA binding molecule which binds to a target RNA molecule in a manner modulatable by a RNA-binding ligand, wherein said RNA-binding ligand and said RNA-binding molecule are different, said method comprising; providing a target RNA molecule; (a) contacting the target RNA molecule with a RNA-binding ligand, to produce a RNA-ligand complex; (b) assessing the ability of candidate RNA-binding molecules to bind the target RNA molecule and the XNA-ligand complex: and isolating those candidate RNA-binding molecules which bind the target RNA molecule and RNA-ligand complex with different binding affinities.

It is further envisaged that the methods of the invention may be advantageously used to select nucleic acid sequences which allow binding of a particular ligand/nucleic acid binding molecule combination, or alternatively or to select polypeptide sequences which allow binding of a particular ligand/polypeptide binding protein combination. For example, one may wish to isolate particular nucleic acid sequences to which a given nucleic acid binding molecule is able to bind, or particular polypeptide sequences to which a given polypeptide binding molecule is able to bind, or to isolate only those nucleic acid or polypeptide sequences which depend on the presence of ligand for the nucleic acid or polypeptide binding molecule to associate with them.

Accordingly, we describe a method for isolating target nucleic acid sequences to which a particular nucleic acid binding molecule will bind, said method comprising providing a library of target nucleic acid molecule(s); contacting said nucleic acid molecules with a nucleic acid binding molecule in the presence or absence of ligand assessing the ability of the candidate target nucleic acid molecule(s) to bind the nucleic acid binding molecule; and isolating those target nucleic acid molecules which bind the nucleic acid binding molecule. We also describe a method for isolating target polypeptide sequences to which a particular polypeptide binding molecule will bind, said method comprising providing a library of target polypeptide molecule(s); contacting said polypeptide molecules with a polypeptide binding molecule in the presence or absence of liqand assessing the ability of the candidate target polypeptide molecule(s) to bind the polypeptide binding molecule: and isolating those target polypeptide molecules which bind the polypeptide binding molecule.

A library of target nucleic acid or polypeptide molecule(s) according to the invention may preferably comprise a plurality of different nucleic acid or polypeptide molecules: preferably said nucleic acid or polypeptide molecules may be related to one another in terms of sequence homology.

A library of candidate nucleic acid or polypeptide binding molecule(s) according to the invention may preferably comprise a plurality of different candidate nucleic acid or polypeptide binding proteins; preferably said candidate nucleic acid or polypeptide binding proteins may be related to one another in terms of amino acid sequence homology.

It is envisaged that this method could be advantageously used in order to isolate nucleic acid or polypeptide sequences which require ligand to associate with a known nucleic acid or polypeptide binding molecule. For example, there may be a nucleic acid or polypeptide sequence which is bound by a known nucleic acid or polypeptide binding molecule in a ligand-independent manner, and it may be desirable to find a nucleic acid or polypeptide sequence(s) which can also associate with the same wild-type nucleic acid or polypeptide binding molecule, but which do so in a ligand-modulatable manner. Preferably, this may be accomplished according to the above method of the present invention.

F. Uses

The assay methods of the invention may be used to identify nucleic acid or polypeptide binding molecules, ligands and/or target nucleic acid or polypeptide where the binding the binding molecule to the target is modulatable by the ligand.

These components, such as nucleic acid binding proteins according to the invention and identified by the assay methods of the invention may be used individually or in combination in a wide variety of applications.

Thus, nucleic acid or polypeptide binding proteins according to the invention and identified by the assay methods of the invention may be employed in a wide variety of applications, including diagnostics and as research tools. Advantageously, they may be employed as diagnostic tools for identifying the presence of particular nucleic acid or polypeptide molecules in a complex mixture. Nucleic acid or polypeptide binding molecules according to the invention can preferably differentiate between different target nucleic acid or polypeptide molecules, and their binding affinities for the nucleic acid or polypeptide target sequences are preferably modulated by ligand(s). Nucleic acid or polypeptide binding molecules according to the invention are useful in switching or modulating gene expression, especially in gene therapy applications and agricultural biotechnology applications as described below.

Specifically, targeted nucleic acid or polypeptide binding molecules, such as zinc fingers, according to the invention may moreover be employed in the regulation of gene transcription, for example by specific cleavage of nucleic acid sequences using a fusion polypeptide comprising a zinc finger targeting domain and a nucleic acid cleavage domain, or by fusion of an transcriptional effector domain to a zinc finger, to activate or repress transcription from a gene which possesses the zinc finger binding sequence in its upstream sequences.

A polypeptide binding protein according to the invention fused to a transcriptional effector domain may be used to target proteins bound to particular gene regulatory sequences such as promoters or enhancers, to turn on or off transcription of a gene. Gene transcription may also be increased or decreased from a promoter or enhancer containing zinc finger binding sequences, by making use of a fusion protein comprising a zinc finger fused to a polypeptide binding protein and another fusion protein comprising a protein which binds to the polypeptide binding protein fused to a transcriptional effector domain. for example. VP16.

Preferably, activation or repression only occurs in the presence of the ligand, since in a preferred embodiment the zinc fingers or polypeptide binding proteins will not bind their target sequences in the absence of the ligand. Alternatively, activation only occurs in the absence of the ligand, since the zinc fingers or polypeptide binding proteins may not bind their target nucleic acid or polypeptide sequences in the presence of the ligand. Zinc fingers capable of differentiating between U and T may be used to preferentially target RNA or nucleic acid, as required. Where RNA-targeting polypeptides are intended,, these are included in the term “nucleic acid binding molecule”.

Thus nucleic acid or polypeptide binding molecules according to the invention will typically require the presence of a transcriptional effector domain, such as an activation domain or a repressor domain. Examples of transcriptional activation domains include the VP16 and VP64 transactivation domains of Herpes Simplex Virus. Alternative transactivation domains are various and include the maize C1 transactivation domain sequence (Sainz et al. 1997, Mol. Cell. Biol. 17: 115-22) and P1 (Goffet al. 1992, Genes Dev. 6: 864-75; Estruch et al. 1994, Nucleic Acids Res. 22: 3983-89) and a number of other domains that have been reported from plants (see Estruch et al., 1994, ibid).

Instead of incorporating a transactivator of gene expression, a repressor of gene expression can be fused to the nucleic acid binding protein or polypeptide binding protein and used to down regulate the expression of a gene contiguous or incorporating the nucleic acid binding protein target sequence, or a gene bound by the target polypeptide of the polypeptide binding protein as described above. Such repressors are known in the art and include, for example, the KRAB-A domain (Moosmann et al. Biol. Chem. 378: 669-677 (1997)) the engrailed domain (Han et al. Embo J. 12: 2723-2733 (1993)) and the snag domain (Grimes et al. Mol Cell. Biol. 16: 6263-6272 (1996)). These can be used alone or in combination to down-regulate gene expression.

Another possible application is the use of zinc fingers fused to nucleic acid cleavage moieties, such as the catalytic domain of a restriction enzyme, to produce a restriction enzyme capable of cleaving only target nucleic acid of a specific sequence (see Kim et al. (1996) Proc. Natl. Acad. Sci. USA 93:11 56-1160). Using such approaches. different nucleic acid binding domains can be used to create restriction enzymes with any desired recognition nucleotide sequence, but which cleave nucleic acid conditionally dependent on the presence or absence of a particular ligand, for instance Distamycin A. It may also be possible to use enzymes other than those that cleave nucleic acids for a variety of purposes.

In a preferred embodiment, the zinc finger polypeptides of the invention may be employed to detect the presence to absence of a particular target nucleic acid sequence in a sample. Similarly, the polypeptide binding proteins of the invention may be used to detect the presence or absence of a particular target polypeptide sequence in a sample.

We therefore describe a method for determining the presence of a target nucleic acid molecule, comprising the steps of: (a) preparing a nucleic acid binding protein by the method set forth above which is specific for the target nucleic acid molecule; (b) exposing a test system which may comprise the target nucleic acid molecule to the nucleic acid binding protein under conditions which promote binding, and removing any nucleic acid binding protein which remains unbound; (c) detecting the presence of the nucleic acid binding protein in the test system. To detect the presence of a target protein in a sample, the following steps may be taken: (a) preparing a polypeptide binding protein by the method set forth above which is specific for the target polypeptide molecule; (b) exposing a test system which may comprise the target polypeptide molecule to the polypeptide binding protein under conditions which promote binding, and removing any polypeptide binding protein which remains unbound; (c) detecting the presence of the polypeptide binding protein in the test system.

Regulation of Gene Expression In Vivo

In a particularly preferred embodiment of the present invention, nucleic acid binding molecules capable of binding to a target nucleic acid in a manner modulatable by a ligand, as well as the polypeptide binding molecules capable of binding to a target polypeptide in a manner modulatable by a ligand, are used to regulate expression from a gene in vivo.

The target gene may be endogenous to the genome of the cell or may be heterologous However, in either case it will comprise a target nucleic acid sequence, such as a target nucleic acid sequence described above, to which a nucleic acid binding molecule of the invention binds in a manner modulatable by a ligand, or which is bound by the complex consisting of a polypeptide and a polypeptide binding protein. Where the nucleic acid binding molecule is a polypeptide, it may typically be expressed from a nucleic acid construct present in the host cell comprising the target sequence. A polypeptide binding protein may similarly be expressed. Such a nucleic acid construct is preferably stably integrated into the genome of the host cell, but this is not essential.

Thus in the case of polypeptide nucleic acid binding molecules, a host cell according to the invention comprises a target nucleic acid sequence and a construct capable of directing expression of the nucleic acid binding molecule in the cell. If a polypeptide binding protein is used, the host cell may comprise a target nucleic acid sequence and a construct capable of directing expression of the polypeptide binding molecule in the cell.

Suitable constructs for expressing the nucleic acid or polypeptide binding molecule are known in the art and are described in section B above. The coding sequence may be expressed constitutively or be regulated. Expression may be ubiquitous or tissue-specific. Suitable regulatory sequences are known in the art and are also described in section B above. Thus the nucleic acid construct will comprise a nucleic acid sequence encoding a nucleic acid binding molecule or a or polypeptide binding molecule operably linked to a regulatory sequence capable of directing expression of the nucleic acid or polypeptide binding molecule in a host cell.

It may also be desirable to use target nucleic acid sequences that include operably linked neighbouring sequences that bind transcriptional regulatory proteins, such as transactivators. Preferably the transcriptional regulatory proteins are endozenous to the cell. If not they typically will need to be introduced into the host cell using suitable nucleic acid constructs.

Techniques for introducing nucleic acid constructs into host cells are known in the art for both prokaryotic and eukaryotic cells, including yeast, fungi, plant and animal cells.

Many of these techniques are mentioned below in the section on the production of transgenic organisms.

Regulation of expression of the gene of interest which comprises a second coding sequence operably linked to the target nucleic acid sequence is typically achieved by administering to the cell a ligand according to the invention. Typically, the ligand is a molecule such as Distamycin A which may be administered exogenously to the cell and taken up by the cell whereupon it may contact the nucleic acid or polypeptide binding molecule and modulate its binding directly or indirectly to the target sequence. For example, two proteins may interact in such a way that they bind to the target sequence only when bound to each other (i.e., dimerised), in which case an antibody which modulates the interaction between two protein binding partners may be used to modulate binding of the proteins to the target sequence. Such antibody ligands may be identified by screening a library of randomised antibodies with the methods of our invention. However polypeptide ligands may also be introduced into the cell either directly or by introducing suitable nucleic acid vectors, including viruses.

The target nucleic acid sequence and the nucleic acid construct encoding the nucleic acid or polypeptide binding molecule are preferably stably integrated into the genome of the host cell. Where the host cell is a single celled organism or part of a multicellular organism, the resulting organism may be termed transgenic. The target nucleic acid may in a preferred embodiment, be a naturally occurring sequence for which a corresponding nucleic acid or polypeptide binding molecule and ligand have been identified using the screening methods of the invention.

The term “multicellular organism” here denotes all multicellular plants, fungi and animals except humans, i.e., prokaryotes and unicellular eukaryotes are excluded specifically. The term also includes an individual organism in all stages of development, including embryonic and fetal stages. A “transgenic” multicellular organisms is any multicellular organism containing cells that bear genetic information received, directly or indirectly, by deliberate genetic manipulation at the subcellular level, such as by microinjection or infection with recombinant virus. Preferably, the organism is transgenic by virtue of comprising at least a heterologous nucleotide sequence encoding a nucleic acid binding molecule (or a polypeptide binding molecule) or target nucleic acid as herein defined.

“Transgenic” in the present context does not encompass classical crossbreeding or in vitro fertilization, but rather denotes organisms in which one or more cells receive a recombinant nucleic acid molecule. Transgenic organisms obtained by subsequent classical crossbreeding or in vitro fertilization of one or more transgenic organisms are included within the scope of the term “transgenic”.

The term “germline transgenic organism” refers to a transgenic organism in which the genetic information has been taken up and incorporated into a germline cell, therefore conferring the ability to transfer the information to offspring. If such offspring, in fact, possess some or all of that information, then they, too, are transgenic multicellular organisms within the scope of the present invention.

The information to be introduced into the organism is preferably foreign to the species of animal to which the recipient belongs (i.e., “heterologous”), but the information may also be foreign only to the particular individual recipient, or genetic information already possessed by the recipient. In the last case, the introduced gene may be differently expressed than is the native gene.

“Operably linked” refers to polynucleotide sequences which are necessary to effect the expression of coding and non-coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and a transcription termination sequence. The term “control sequences” is intended to include, at a minimum, components whose presence can influence expression, and can also include additional components whose presence is advantageous, for example, leader sequences and fusion partner sequences.

Since the nucleic acid constructs are typically to be integrated into the host genome. it is important to include sequences that will permit expression of polypeptides in a particular genomic context. One possible approach would to use homologous recombination to replace all or part of the endogenous gene whose expression it is desired to regulate with equivalent sequences comprising a target nucleic acid in its regulatory sequences. This should ensure that the gene is subject to the same transcriptional regulatory mechanisms as the endogenous gene, with the exception of the target nucleic acid sequence. Alternatively, homologous recombination may be used in a similar manner but with the regulatory sequences also replaced so that the gene is subject to a different form of regulation.

However, if the construct encoding either the nucleic acid binding molecule (or polypeptide binding molecule) or target nucleic acid is placed randomly in the genome, it is possible that the chromatin in that region will be transcriptionally silent and in a condensed state. If this occurs, then the polypeptide will not be expressed—these are termed position-dependent effects. To overcome this problem, it may be desirable to include locus control regions (LCRs) that maintain the intervening chromatin in a transcriptionally competent open conformation. LCRs (also known as scaffold attachment regions (SARs) or matrix attachment regions (MARs)) are well known in the art—an example being the chicken

Ivsozyme A element (Stief et al., 1989. Nature 341: 343). which can be positioned around an expressible gene of interest to effect an increase in overall expression of the gene and diminish position dependent effects upon incorporation into the organism s genome (Stief et al. 1989, supra). Another example is the CD2 gene LCR described by Lang et al., 1991, Nucl. Acid. Res. 19: 5851-5856.

Thus, a polynucleotide construct for use in the present invention, to introduce a nucleotide sequence encoding a nucleic acid or polypeptide binding molecule into the genome of a multicellular organism, typically comprises a nucleotide sequence encoding the nucleic acid or polypeptide binding molecule operably linked to a regulatory sequence capable of directing expression of the coding sequence. In addition the polynucleotide construct may comprise flanking sequences homologous to the host cell organism genome to aid in integration. An alternative approach would be to use viral vectors that are capable of integrating into the host genome, such as retroviruses.

Preferably, a nucleotide construct for use in the present invention further comprises flanking LCRs.

Construction of Transgenic Organisms Expressing Nucleic Acid Binding Molecules

A transgenic organism of the invention is preferably a multicellular euklaryotic organism, such as an animal, a plant or a fungus. Animals include animals of the phyla cnidaria ctenophora, platyhelminthes, nematoda, annelids mollusca, chelicerata, uniramia crustacea and chordata. Uniramians include the subphylum hexpoda that includes insects such as the winged insects. Chordates includes vertebrate groups such as mammals, birds. reptiles and amphibians. Particular examples of mammals include non-human primates, cats, dogs, ungulates such as cows, goats, pigs, sheep and horses and rodents such as mice, rats, gerbils and hamsters.

Plants include the seed-bearing plants (angiosperms) and conifers. Angiosperms include dicotyledons and monocotyledons. Examples of dicotyledonous plants include tobacco. (Nicotiana plumbaginifolia and Nicotiana tabacum), arabidopsis (Arabidopsis thaliaina), Brassica napus, Brassica nigra, Datura innoxia, Vicia narbonensis, Vicia jaba, pea (Pisum saizivum), cauliflower, carnation and lentil (Letis culinaris). Examples of monocotyledonous plants include cereals such as wheat, barley, oats and maize.

Production of Transgenic Animals

Techniques for producing transgenic animals are well known in the art. A useful general textbook on this subject is Houdebine, Transgenic animals—Generation and Use (Harwood Academic, 1997)—an extensive review of the techniques used to generate transgenic animals from fish to mice and cows.

Advances in technologies for embryo micromanipulation now permit introduction of heterologous nucleic acid into, for example, fertilized mammalian ova. For instance. totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired nucleic acid, and transgenic animals produced from the infected embryo. In a most preferred method, however, the appropriate nucleic acids are conjected into the pronucleus or cytoplasm of embryos, preferably at the single cell stage. and the embryos allowed to develop into mature transgenic animals. Those techniques as well known. See reviews of standard laboratory procedures for microinjection of heterologous nucleic acids into mammalian fertilized ova, including Hogan et al., Manipulating the Mouse Embryo, (Cold Spring Harbor Press 1986); Krimpenfort et al. Bio/Technology 9:844 (1991); Palmiter et al., Cell. 41: 343 (1985); Kraemer et al., Genetic manipulation of the Mammalian Embryo, (Cold Spring Harbor Laboratory Press 1985); Hammer et al. Nature. 315: 680 (1985); Wagner et al. U.S. Pat. No. 5,175,385; Krimpenfort et al., U.S. Pat. No. 5,175384, the respective contents of which are incorporated herein by reference

Another method used to produce a transgenic animal involves microinjecting a nucleic acid into pro-nuclear stage ecas by standard methods. Injected eggs are then cultured before transfer into the oviducts of pseudopregnant recipients.

Transgenic animals may also be produced by nuclear transfer technology as described in Schnieke, A. E, et al. 1997. Science. 278: 2130 and Cibelli. J. B, et al. 1998, Science. 280: 1256. Using this method, fibroblasts from donor animals are stably transfected with a plasmid incorporating the coding sequences for a binding domain or binding partner of interest under the control of regulatory. Stable transfectants are then fused to enucleated oocytes, cultured and transferred into female recipients.

Analysis of animals which may contain transgenic sequences would typically be performed by either PCR or Southern blot analysis following standard methods.

By way of a specific example for the construction of transgenic mammals, such as cows, nucleotide constructs comprising a sequence encoding a nucleic acid binding molecule are microinjected using, for example, the technique described in U.S. Pat. No. 4,873.191. into oocytes which are obtained from ovaries freshly removed from the mammal. The oocytes are aspirated from the follicles and allowed to settle before fertilization with thawed frozen sperm capacitated with heparin and prefractionated by Percoll gradient to isolate the motile fraction.

The fertilized oocytes are centrifuged, for example, for eight minutes at 15.000 g to visualize the pronuclei for injection and then cultured from the zygote to morula or blastocyst stage in oviduct tissue-conditioned medium. This medium is prepared by using luminal tissues scraped from oviducts and diluted in culture medium. The zygotes must be placed in the culture medium within two hours following microinjection.

Oestrous is then synchronized in the intended recipient mammals, such as cattle, by administering coprostanol. Oestrous is produced within two days and the embryos are transferred to the recipients 5-7 days after estrous. Successful transfer can be evaluated in the offspring by Southern blot.

Alternatively, the desired constructs can be introduced into embryonic stem cells (ES cells) and the cells cultured to ensure modification by the transgene. The modified cells are then injected into the blastula embryonic stage and the blastulas replaced into pseudopregnant hosts. The resulting offspring are chimeric with respect to the ES and host cells, and nonchimeric strains which exclusiveiv comprise the ES progeny can be obtained using conventional cross-breeding. This technique is described, for example, in WO91/10741.

Production of Transgenic Plants

Techniques for producing transgenic plants are well known in the art. Typically, either whole plants, cells or protoplasts may be transformed with a suitable nucleic acid construct encoding a nucleic acid binding molecule or a polypeptide binding molecule or target nucleic acid (see above for examples of nucleic acid constructs). There are many methods for introducing transforming nucleic acid constructs into cells, but not all are suitable for delivering nucleic acid to plant cells. Suitable methods include Agrobacterium infection (see, among others. Turpen et al. 1993, J. Virol. Methods, 42: 227-239) or direct delivery of nucleic acid such as, for example, by PEG-mediated transformation, by electroporation or by acceleration of nucleic acid coated particles. Acceleration methods are generally preferred and include, for example, microprojectile bombardment. A typical protocol for producing transgenic plants (in particular moncotyledons), taken from U.S. Pat. No. 5,874,265, is described below.

An example of a method for delivering transforming nucleic acid segments to plant cells is microprojectile bombardment. In this method, non-biological particles may be coated with nucleic acids and delivered into cells by a propelling force. Exemplary particles include those comprised of tungsten, gold, platinum, and the like.

A particular advantage of microprojectile bombardment, in addition to it being an effective means of reproducibly stably transforming both dicotyledons and monocotyledons, is that neither the isolation of protoplasts nor the susceptibility to Agrobacterium infection is required. An illustrative embodiment of a method for delivering nucleic acid into plant cells by acceleration is a Biolistics Particle Delivery System, which can be used to propel particles coated with nucleic acid through a screen, such as a stainless steel or Nytex screen, onto a filter surface covered with plant cells cultured in suspension. The screen disperses the tungsten-nucleic acid particles so that they are not delivered to the recipient cells in large aggregates. It is believed that without a screen intervening between the projectile apparatus and the cells to be bombarded, the projectiles aggregate and may be too large for attaining a high frequency of transformation. This may be due to damage inflicted on the recipient cells by projectiles that are too large.

For the bombardment, cells in suspension are preferably concentrated on filters. Filters containing the cells to be bombarded are positioned at an appropriate distance below the macroprojectile stopping plate. If desired, one or more screens are also positioned between the gun and the cells to be bombarded. Through the use of techniques set forth herein one may obtain up to 1000 or more clusters of cells transiently expressing a marker gene (“foci”) on the bombarded filter. The number of cells in a focus which express the exogenous gene product 48 hours post-bombardment often range from 1 to 10 and average 2 to 3.

After effecting delivery of exogenous nucleic acid to recipient cells by any of the methods discussed above, a preferred step is to identify the transformed cells for filer culturing and plant regeneration. This step may include assaying cultures directly for a screenable trait or by exposing the bombarded cultures to a selective agent or agents.

An example of a screenable marker trait is the red pigment produced under the control of the R-locus in maize. This pigment may be detected by culturing cells on a solid support containing nutrient media capable of supporting growth at this stage, incubating the cells at, e.g., 18° C, and greater than 180 μE m⁻²s⁻¹, and selecting cells from colonies (visible aggregates of cells) that are pigmented. These cells may be cultured further, either in suspension or on solid media.

An exemplary embodiment of methods for identifying transformed cells involves exposing the bombarded cultures to a selective agents such as a metabolic inhibitor, an antibiotic, herbicide or the like. Cells which have been transformed and have stably integrated a marker gene conferring resistance to the selective agent used will grow and divide in culture. Sensitive cells will not be amenable to further culturing.

To use the bar-bialaphos selective system, bombarded cells on filters are resuspended in nonselective liquid medium, cultured (e.g., for one to two weeks) and transferred to filters overlaying solid medium containing from 1-3 mg/l bialaphos. While ranges of 1-3 mg/l will typically be preferred, it is proposed that ranges of 0.1-50 mg/l will find utility in the practice of the invention. The type of filter for use in bombardment is not believed to be particularly crucial, and can comprise any solid, porous, inert support.

Cells that survive the exposure to the selective agent may be cultured in media that supports regeneration of plants. Tissue is maintained on a basic media with hormones for about 2-4 weeks, then transferred to media with no hormones. After 2-4 weeks, shoot development will signal the time to transfer to another media.

Regeneration typically requires a progression of media whose composition has been modified to provide the appropriate nutrients and hormonal signals during sequential developmental stages from the transformed callus to the more mature plant. Developing plantlets are transferred to soil, and hardened, e.g., in an environmentally controlled chamber at about 85% relative humidity, 600 ppm CO₂, and 250 μE m⁻²s⁻¹ of light Plants are preferably matured either in a growth chamber or greenhouse. Regeneration will typically take about 3-12 weeks. During regeneration, cells are grown on solid media in tissue culture vessels. An illustrative embodiment of such a vessel is a petri dish. Regenerating plants are preferably grown at about 19° C., to 28° C. After the regenerating plants have reached the stage of shoot and root development, they may be transferred to a greenhouse for further growth and testing.

Genomic DNA may be isolated from callus cell lines and plants to determine the presence of the exogenous gene through the use of techniques well known to those skilled in the art such as PCR and/or Southern blotting.

Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27).

Thus, in one aspect, the present invention relates to a vector system which carries a construct encoding a nucleic acid or polypeptide binding molecule or target nucleic acid according to the present invention and which is capable of introducing the construct into the genome of an organism, such as a plant.

The vector system may comprise one vector, but it can comprise at least two vectors. In the case of two vectors, the vector system is normally referred to as a binary vector system. Binary vector systems are described in further detail in Gynheung An et al. (1980). Binary Vectors, Plant Molecular Biology Manual A3. 1-19.

One extensively employed system for transformation of plant cells with a given promoter or nucleotide sequence or construct is based on the use of a Ti plasmid from Agrobacterium tumefaciens or a Ri plasmid from Agrobacterium rhizogenes (An et al. (1986), Plant Physiol. 81. 301-305 and Butcher D. N, et al. (1980), Tissue Culture Methods for Plant Pathologists, eds.: D. S. Ingrams and J. P. Helgeson, 203-208).

Several different Ti and Ri plasmids have been constructed which are suitable for the construction of the plant or plant cell constructs described above.

Examples of Specific Applications

The nucleic acid (or polypeptide) binding molecule/ target nucleic acid (or polypeptide)/ligand combination may be used to regulate the expression of a nucleotide sequence of interest, such as in a cell of an organism, including prokaryotes, yeasts, flini, plants and animals, for example mammals, including humans.

Nucleotide sequences of interest include genes associated with disease in humans and animals and therapeutic genes. Thus a nucleic acid or polypeptide binding molecule may be used in conjunction with a target nucleic acid or polypeptide sequence and ligand in a method of treating or preventing disease in an animal or human patient.

Alternatively, a switching system, whether a gene switch or a protein switch, may be used to regulate expression of a nucleotide sequence of interest in a plant. Examples of specific applications include the following:

1. Improvement of ripening characteristics in fruit. A number of genes have been identified that are involved in the ripening process (such as in ethylene biosynthesis). Control of the ripening process via regulation of the expression of those genes will help reduce significant losses via spoilage.

2. Modification of plant growth characteristics through intervention in hormonal pathways. Many plant characteristics are controlled by hormones. Regulation of the genes involved in the production of and response to hormones will enable produce crops with altered characteristics.

3. Improvement of other characteristics by manipulation of plant gene expression. Overexpression of the Na+/H+ antiport gene has resulted in enhanced salt tolerance in Arabidopsis. Targetted zinc fingers could be used to regulate the endogenous gene.

4. Improvement of plant aroma and flavour. Pathways leading to the production of aroma and flavour compounds in vegetables and fruit are currently being elucidated allowing the enhancement of these traits using gene switch technology.

5. Improving the pharmaceutical and nutraceutical potential of plants. Many pharmaceutically active compounds are known to exist in plants but in many cases production is limited due to insufficient biosynthesis in plants. Gene switch technology could be used to overcome this limitation by upregulating specific genes or biochemical pathways. Other uses include regulating the expression of genes involved in biosynthesis of commercially valuable compounds that are toxic to the development of the plant.

6. Reducing harmful plant components. Some plant components lead to adverse allergic reaction when ingested in food. Gene switch technology could be used to overcome this problem by downregulating specific genes responsible for these reactions.

7. As well as modulating the expression of endogenous genes, heterologous genes may be introduced whose expression is regulated by a gene switch of the invention. For example, a nucleotide sequence of interest may encode a gene product that is preferentially toxic to cells of the male or female organs of the plant such that the ability of the plant to reproduce can be regulated. Alternatively, or in addition, the regulatory sequences to which the nucleotide sequence is operably linked may be tissue-specific such that expression when induced only occurs in male or female organs of the plant. Suitable sequences and/or gene products are described in WO8911 0396, WO92/04454 (the TA29 promoter from tobacco) and EP-A-344.029, EP-A412,006 and EP-A-412.91 1.

Other uses include regulating the expression of genes involved in biosynthesis of commercially valuable compounds that are toxic to the development of the plant.

The present invention will now be described by way of the following examples. which are illustrative only and non-limiting. The examples refer to the figures:

### BRIEF DESCRIPTION OF THE FIGURES

FIG. 1 shows a graph of the effect of Distamycin A concentration on binding of two different phage (clone 3 (3/2F) and clone 4 (4/EF)) to the DNA sequence AAAAAGGCG. In this case, the small molecule causes phage binding to nucleic acid.

FIG. 2 shows a graph of the effect of Actinomycin D concentration on binding of two different phage (AD clone 1 and 6) to the DNA sequence AGCTTGGCG. In this case. the small molecule causes phage binding to nucleic acid.

FIG. 3 shows four different phage (0.4/1, 0.4/2, 0.4/4 and 0.4/5) binding to the randomised DNA oligo YRYRYGGCG (where Y is C or T and R is G or A) in the presence, but not in the absence, of echinomycin (EM).

FIG. 4 shows the binding site signature of phage 0.4/4 selected using the randomised DNA sequence (Y1)(R2)(Y3)(R4)(Y5)GGCG. The phage has a preference for the DNA sequence (T)(G/A)(C)(G/A)(T) in the presence of echinomycin.

FIG. 5 shows binding of the phage 0.4/4 to three related DNA sequences. TACGTGGCG, TGTATGGCG and CGTACGGCG, as a function of echinomycin concentration. The first DNA site contains the optimal binding sequence as revealed by the binding site signature.

FIG. 6 shows a graph of the effect of ligand concentration on binding of two different phage to specific DNA sequences. In this case, the respective phage are dissociated from the DNA in the presence of distamycin A or actinomycin D.

### EXAMPLES

### Example 1

### Preparation and Screening of a Zinc Finger Phage Display Library

Selection of Zinc Finger Phage Binding DNA Targets in the Presence of Small Molecules

### Example 1.1

### Selection of Zinc Finger Phage that Bind DNA in the Presence of Distamycin A

A powerful method of selecting DNA binding proteins is the cloning of peptides (Smith (1985) Science 228, 1315-1317), or protein domains (McCafferty et al. (1 990) Nature 348:552-554: Bass et al., (1990) Proteins 8:309-314), as fusions to the minor coat protein (pm) of bacteriophage fd, which leads to their expression on the tip of the capsid. A phage display library is created comprising variants of the middle finger from the DNA binding domain of ZifP68.

Materials and Methods

Construction and Cloning of Genes.

In general, procedures and materials are in accordance with guidance given in Sambrook et al., Molecular Cloning. A Laboratory Manual, Cold Spring Harbor, 1989. The gene for the Zif268 fingers (residues 333420) is assembled from 8 overlapping synthetic oligonucleotides (see Choo and Klug, (1994) PNAS (USA) 91:11163-67), giving SfiI and NotI overhangs. The genes for fingers of the phage library are synthesised from 4 oligonucleotides by directional end to end ligation using 3 short complementary linkers, and amplified by PCR from the single strand using forward and backward primers which contain sites for NotI and SfiI respectively. Backward PCR primers in addition introduce Met-Ala-Glu as the first three amino acids of the zinc finger peptides, and these are followed by the residues of the wild type or library fingers as required. Cloning overhangs are produced by digestion with SfiI and NotI where necessary. Fragments are ligated to 1 μg similarly prepared Fd-Tet-SN vector. This is a derivative of fd-tet-DOG 1 (Hoogenboom et al., (1991) Nucleic Acids Res. 19,41334137) in which a section of the pelB leader and a restriction site for the enzyme SfiI (underlined) have been added by site-directed mutagenesis using the oligonucleotide:

5′ CTCCTGCAGTTGGACCTGTGCCATGGCCGGCTGGGCCGCATAGAATGG AACAACTAAAGC 3′ (Seq ID No. 1)

which anneals in the region of the polylinker. Electrocompetent DH5α cells are transformed with recombinant vector in 200 ng aliquots, grown for 1 hour in 2× TY medium with 1% glucose and plated on TYE containing 15 μg/ml tetracycline and 1% glucose.

The zinc finger phage display library of the present invention contains amino acid randomisations in putative base-contacting positions from the second and third zinc fingers of the three-finger DNA binding domain of Zif)68, and contains members that bind DNA of the sequence XXXXXGGCG where X is any base. Further details of the library used may be found in WO 98/53057, which is incorporated herein by reference. The DNA sequences AAAAAAGGCG and AAAAAAGGCGAAAAAA are used as selection targets in this example because short runs of adenines can cause intrinsic DNA bending moreover, the structure of the bend can be disrupted by binding of the antibiotic distamycin A.

Phage Selection.

Bacterial colonies containing zinc finger phage libraries are transferred from plates to 200 ml 2× TY medium (16 g/liter Bactotryptone, 10 g/liter Bactoyeast extract. 5 g/liter NaCl) containing 50 μM ZnCl₂ and 15 μg/ml tetracycline. Bacterial cultures are grown overnight at 30° C. Culture supernatant containing phages is obtained by centrifuging at 1500× g for D minutes.

Phage selection is over 4 rounds. Before each round, a pre-selection step is included comprising binding of 10 μmol of biotinylated DNA target sites immobilised on 50 mg streptavidin coated beads (Dynal) to 1 ml of phage solution (bacterial culture supernatant diluted 1:1 with PBS containing 50 μM ZnCl₂, 4% Marvel, 2% Tween), for 1 hour at 20° C. on a rolling platform. After this time, 0.5 ml of phage solution is transferred to a streptavidin coated tube and incubated with 2 pmol biotinylated DNA target site in the presence of 2 μM distamycin A (Sigma) and 4 μg poly [d(I−C)]. After a one hour incubation the tubes are washed 20 times with PBS containing 50 μM ZnCl₂ and 1% Tween, and 3 times with PBS containing 50 μM ZnCl₂. Phage are eluted using 0.1 ml 0.1 M triethylamine and the solution is neutralised with an equal volume of 1M Tris-Cl (pH 7.4). Logarithmic-phase E. coli TG1 cells are infected with eluted phage, and grown overnight. as described above, to prepare phase supernatants for subsequent rounds of selection.

After 4 rounds of selection, bacteria are plated and phage prepared from 96 colonies are screened for binding to the DNA target site in the presence and absence of distamycin A. Binding reactions are carried out in wells of a streptavidin-coated microtitre plate (Boehringer alannheim) and contain 50 μl of phage solution (bacterial culture supernatant diluted 1:1 , with PBS containing 50 μM ZnCl₂, 4% Marvel. 2% Tween). 0.1 5 μmol DNA target site and 0.25 μg poly [d(I−C)]. When added, distamycin A is present at a concentration of 2 μM. After a one hour incubation the wells are washed 20 times with. PBS containing 50 μM ZnCl₂ and 1% Tween (and also distatmycin A at a concentration of 2 μM where appropriate), and 3 times with PBS containing 50 μM ZnCl). Bound phage are detected by ELISA (carried out in the presence of distamycin A at a concentration of 2 μM where appropriate) with horseradish peroxidase-conjugated anti-M13 IgG (Pharmacia Biotech) and quantitated using SOFTMAX 2.32 (Molecular Devices).

Sequencing of Selected Phage.

Single colonies of transformants obtained after four rounds of selection as described, are Trojan overnight in 2× TY/Zn/Tet. Small aliquots of the cultures are stored in 15% glycerol at −2° C., to be used as an archive. Single-stranded DNA is prepared from phage in the culture supernatant and sequenced using the Sequenase™ 2.0 kit (U.S. Biochemical Corp.). The amino acid sequences of the zinc finger clones are deduced.

Amino Acid Sequences from Helical Regions of Zinc Fingers Selected to Bind DNA in the Presence of Distamycin

| | | | |
|---|---|---|---|
| | F1 | F2 | F3 |
| | −1123456 | −1123456 | −1123456 |
| Clone 1 | RSDELTR | RSDDLST | TNNTRIK |
| Clone 2 | RSDELTR | RSDDLST | HKATRIK |
| Clone 3 | RSDELTR | RSDDLST | TDKVRKK |
| Clone 4 | RSDELTR | RSDDLST | HNASRIN |
| Clone 5 | RSDELTR | RSDDLSV | TNNSRKK |
| Clone 6 | RSDELTR | RSDDLST | TNATRKK |
| Clone 7 | PSDELTR | RSDDLSQ | TRNTRKN |
| Clone 8 | RSDELTR | RSDDLSV | TNNSRKN |

Clones 1-4 were selected to bind the oligo:
tataAAAAAGGCGTGtcacagtcagtccacacgtc
Clones 5-8 were selected to bind the oligo:
tataAAAAAAGGCGAAAAAAtcacagtcagtccacacacgtc

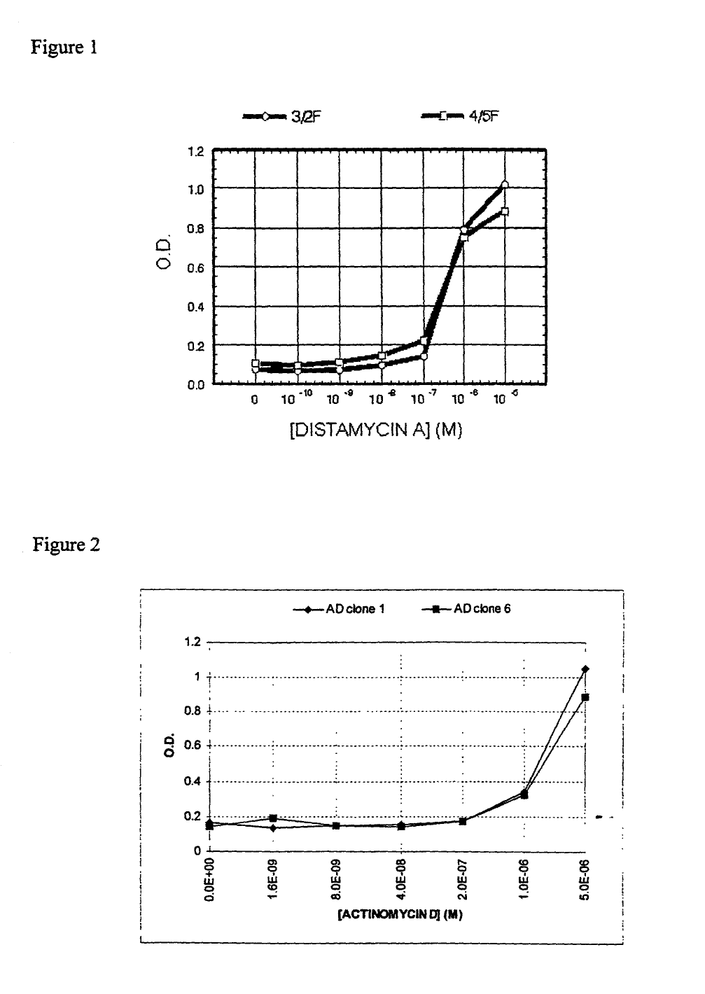

Zinc finger phage clones are isolated according to this method which bind the target with higher affinity in the presence of ligand than in the absence of ligand (see FIG. 1). This method also selected certain clones that bound DNA in the absence of the ligand but were displaced from the DNA in the presence of the ligand (see Example 1.4 below).

### Example 1.2

### Selection of Zinc Finger Phage Binding DNA in the Presence of Actinomycin D

An adaptation to the method outlined in the Example 1.1 was used to isolate phage that bound DNA in the presence of a different small molecule, actinomycin D. In this example the DNA target was AGCTTGGCG.

Phage Selection

Essentially the method was the same as used in the previous section using four rounds of a preselection step followed by a selection step, washing and elution. Differences in the method are described. The preselection step comprised of 7.5 μmol of biotinylated DNA target site immobilised on 18.75 μl streptavidin coated beads (Dynal) in a 100 μl mixture containing 4 μl phage library 96 μl PBS, 2% Marvel. 1% Tween-20. 50 μM ZnCl₂ for 1 hour at room temperature with constant mixing. Phage selections were made in streptavidin coated tubes with the phage supernatant, 5 nM biotinylated target DNA, 10 μM actinomycin D in the presence of 1 μg poly [d(I-C)] competitor. The selections were incubated for 1 hour at room temperature. The bound phage were washed and eluted as described above.

ELISA was performed as described above but using 5 nM biotinylated target DNA. 0.25 μg poly[d(I−C)] competitor in the assay and 10 μM actinomycin D where appropriate. Phage were sequenced using Big Dye Terminator Cycle Sequencing Kit (Perkin Elmer Biosystems) and automated sequencing.

The amino acid sequences from the helical regions of the selected zinc fingers were sequenced as:

| | | | |
|---|---|---|---|
| clone 1 | RSDELTRHIRIH | RSDTLSVHIRTH | HNAHRKTHTKIH |
| clone 6 | RSDELTRHIRIH | RSDHLSVHIRTH | KKFAHSAHRKTHTKIH |

These two clones were selected using the oligo:

tatacaAGCTTGGCGatcacagtcagtccacacptc

These zinc finger clones bind to the target oligo with higher affinity in the presence of actinomycin D than in the absence of ligand (see FIG. 2).

### Example 1.3

### Selection of Zinc Finger Phage Using Randomised DNA in the Presence of Echinomycin, and Subsequent Deconvolution of Binding Partners

In this experiment the library of DNA binding molecules was sorted using a library of DNA sequences in the presence of a small molecule. After DNA binding molecules that bound to DNAs in the presence of the small molecule had been selected, the optimal binding site(s) for each DNA binding molecule were determined using the binding site signature.

a) Selections

In this experiment, 50 μmol of DNA target library of sequence YRYRYGGCG (where Y is C or T and R is G or A) was bound to 125 μl of streptavidin coated beads (Dynal) and the beads were used to preselect 0.4 μl of phage library in 100 μl of PBS, 2% 20 Marvel, 1% Tween-20, 50 μM ZnCl, for 1 hour at room temperature with constant mixing. Phage selections were made in streptavidin coated tubes with the phage supernatant, 30 nM biotinylated target DNA. 10 μM echinomycin in the presence of 1 μg poly [d(I−C)] competitor. The selections were incubated for 1 hour at room temperature. The bound phage were washed and eluted as described above.

ELISA was performed as described above but using 30 μM biotinylated target DNA. 0.5 μg poly[d(I−C)] competitor in the assay and 10 μM echinomycin where appropriate. Phage were sequenced using Big Dye Terminator Cycle Sequencing Kit (Perkin Elmer Biosystems) and automated sequencing.

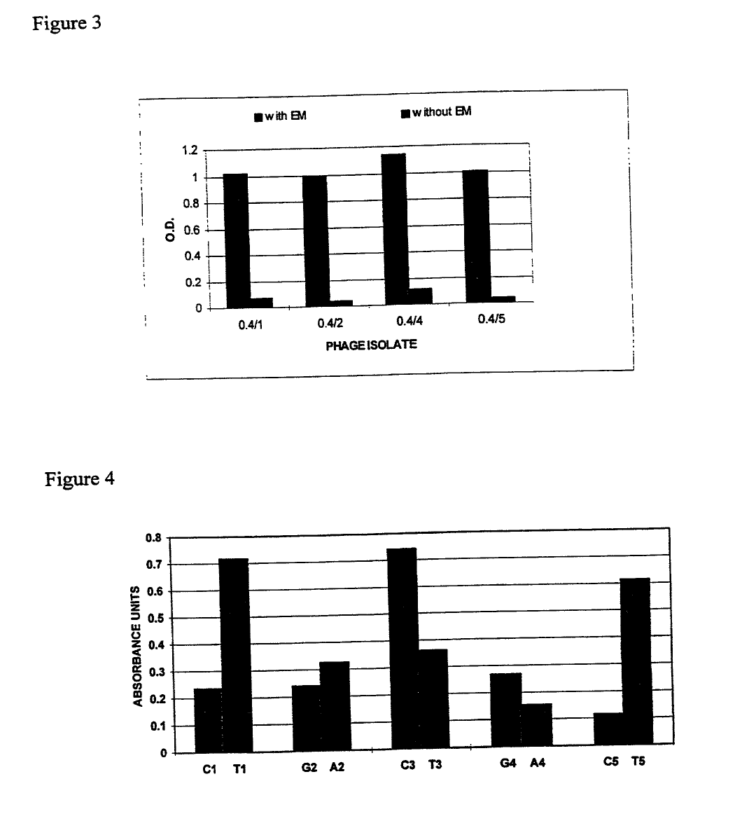

Four different clones were selected using the DNA library tatagtYRYRYGGCG atcacagtcagtccacacgtc in the presence of echinomycin (see FIG. 3).

The amino acid sequences from the helical regions of the selected zinc fingers were sequenced as:

| | | | |
|---|---|---|---|
| clone | RSDELTRHIRIH | RSDHLSKHIRTH | KKFARSQTRINHTKIH |
| 0.4/1 | | | |
| clone | RSDELTRRIRIH | RSDHLSEHIRTH | TRNARTKHTKIH |
| 0.4/2 | | | |
| clone 0.4/4 | RSDELTRHIRIH | RSDHLSNHIRTH | RNDTRKTHTKIH |
| clone 0.4/5 | RSDELTRHIRIH | RSDNLSTHIRTH | KKFAHSNTRKNHTKIH |

b) Binding Site Signature

The signature of the clone 0.4/4 was determined using a modified binding site signature assay. For each of the 5 randomised positions of the oligo, a base was fixed at one of the five positions whilst the remaining 4 positions contained defined mixtures of bases. For the pyrimidine position the base was fixed as either C or T and for the purine position the base was fixed as either G or A so that by testing each position in turn an optimal sequence or binding site signature could be determined.

In each well of a streptavidin-coated microtitre plate 2 p of phage solution (overnight E. coli culture supernatant containing phage) were mixed with 48 μl of 2% Marvel. 1% Tween-20. 0.5 μg poly [d(I−C)], 10 μM echinomycin and between 8-16 μM of biotinylated target DNA. The reaction was incubated for 1 hour at room temperature, followed by 6 washes with PBS containing 1% Tween-20, 50 μM ZnCl₂ and 3 washes with PBS containing 0.05% Tween-20. 50 μM ZnCl₂. 100 μl of PBS containing 1% Marvel, 0.05% Tween-20, 50 μM ZnCl₂ and 1/5000 dilution of anti-M13 horse radish peroxidase antibody conjugate (Amersham Pharmacia Biotech) was added to each well and incubated for 1 hour at room temperature. The ELISA plate was washed 3 times with PBS containing 0.05% Tween-20. 50 μM ZnCl₂ followed by three washes with 3 washes of PBS containing 50 μM ZnCl₂. The assay was developed with BCIP/NBT substrates and quantified using a plate reader.

This method determined the binding site sequence of clone 0.4/4 to be (T₁)(G/A₂)(C₃)(G/A₄)(T₅) (see FIG. 4).

C) Verification of the Target DNA Sequence

The optimal target DNA sequence, as determined by the binding site signature, was synthesised together with two other related DNA sequences that were present in the original random DNA library but differed in some of the optimal base positions of the binding site.

These oligonucleotides had the sequence:

tatagtTACGTGGCGatcacagtcagtccacacgtc
tatagtTGTATGGCGatcacagtcagtccacacgtc
tatagtCGTACGGCGatcacagtcagtccacacgtc

Binding of the phage clone was tested as a function of DNA concentrations (from 5 μM to 0.312 μM) in the presence of 10 μM echinomycin. A phage ELISA was set up using 20 μl phage supernatant, 0.5 μg poly[d(I−C)], 10 μM echinomycin in PBS containing 1% Marvel. 1% Tween-20. 50 μM ZnCl. The total volume of the assay was 50 μl. The assay was washed and developed as described as for the binding site signature assay.

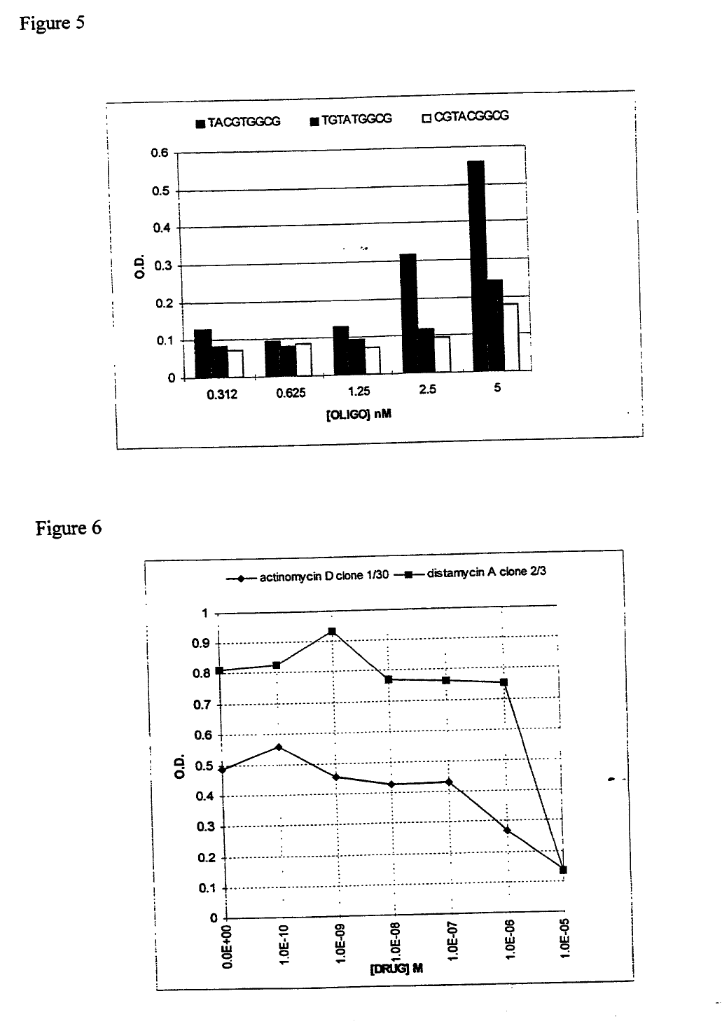

This method showed that the clone 0.4/4 bound preferentially to the sequence determined from the binding site signature, i.e. TACGTGGCG, in the presence of the small molecule (see FIG. 5).

### Example 1.4

### Selection of Zinc Finger Phage that are Dissociated from their DNA Targets in the Presence of Distamycin A or Actinomycin D

This example describes phage that bound DNA targets with higher affinity in the absence of ligand. These phage were isolated using either: (a) the same method as in example 1.1, or (b) by selection in the absence of small molecule and phage elution from DNA using a small molecule.

In this latter case (b) the method was as follows.

Phage selection is over 4 rounds. Binding reactions contain 10 μmol biotinylated DNA site immobilised on 50 mg streptavidin coated beads (Dynal) and a 1 ml solution of ;zinc finger phage library (as described in 1.1) Reactions were incubated for 1 h on a rolling platform. After this time, beads were washed 20 times as described in 1.1 and finally phage were eluted from the beads over 5 minutes using a solution containing ligand (10 μM Distamycin A, or 1 μM Actinomycin D in PBS/Zn).

Some phage isolated by either of the above methods (a or b) bound DNA in the absence of ligand but could be displaced by concentrations of distamycin A at 10 μM and actinomycin D at 1 μM. The distamycin sensitive clone was selected using the DNA target AAAAAGCGGAAAAA and its helices were sequenced as:

QSRSLIQ QRDSLSR RSDERKR

The actinomycin D sensitive clone was selected with the DNA target AGCTTGGCG and its helices were sequenced as:

RSDELTR RSDVLST TRSSRKK

FIG. 6 demonstrates the sensitivity of each clone to the respective drug.

### Example 2

### Modulation of Binding of Polypeptides to Target DNA by Ligand

Individual phage clones are assayed for modulation of target DNA binding by ligand in a phage ELISA binding assay.

Binding assay reactions are carried out in wells of a streptavidin-coated microtitre plate (Boehringer Mannheim) as in Example 1, except that the distamycin concentration is varied while the DNA concentration is kept constant at 2 nM.

Induction of higher affinity DNA binding is observed when distamycin is added to the binding reaction at 10⁻⁶M-10⁻⁷M.

Binding of the zinc finger phage to DNA in the absence of ligand, or at ligand concentrations of 10⁻⁹ M or lower, results in phage retention close to background level, i.e. lower affinity binding than in the presence of ligand.

Background level affinity binding is defined as the phage retention in binding reactions that contain no DNA binding site.

### Example 3

### DNA-Ligand Modulatable Restriction Enzyme

Phage-selected or rationally designed zinc finger domains which bind target DNA sequences in a manner modulatable by a ligand can be converted to restriction enzymes which cleave DNA containing said target sequences in a manner modulatable by ligand. This is achieved by coupling an appropriate zinc finger, as isolated in Example 1 above, to a cleavage domain of a restriction enzyme or other nucleic acid cleaving moiety.

A method of converting zinc finger DNA binding domains to chimaeric restriction endonucleases has been described in Kim, et al. (1996) Proc. Natl. Acad. Sci. USA 93: 1156-1160. In order to demonstrate the applicability of DNA ligand-modulatable zinc fingers to restriction enzymes, a fusion is made between the catalytic domain of Fok I as described by Kim et al, and a zinc finger of Example 1. Fusion of the zinc finger nucleic acid-binding domain to the catalytic domain of Fok I restriction enzyme results in a novel endonuclease which cleaves DNA adjacent to the DNA recognition sequence of the zinc finger (AAAAAAGGCG or AAAAAAGGCGAAAAAA).

The oligonucleotides AAAAAAGGCG and AAAAAAGGCGAAAAAA are synthesised and ligated to arbitrary DNA sequences. After incubation with the zinc finger restriction enzyme, the nucleic acids are analysed by gel electrophoresis. Bands indicating cleavage of the nucleic acid at a position corresponding to the location of the oligonucleotide(s) (AAAAAAGGCG/AAAAAAGGCGAAAAAA) are visible.

In a further experiment, the zinc finger is fused to an amino terminal copper/nickel binding motif. Under the correct redox conditions (Nagaoka M, et al. (1994) J. Am. Chem. Soc. 116:40854086), sequence-specific DNA cleavage is observed, only in the presence of DNA incorporating oligonucleotide AAAAAAGGCG or AAAAAAGGCGAAAAAA.

### Example 4

### Modulation Of Transcriptional Activity In Vivo

A reporter system is produced which produces a reporter signal conditionally depending on the binding of the zinc finger DNA binding molecule to its target DNA sequence. This binding, and hence transcription from the reporter system, is modulated by the ligand Distamycin A.

A transient transfection system using zinc finger transcription factors is produced as described in Choo, Y., et al., (1997) J. Mol. Biol 273:525-532. This system comprises an expression plasmid which produces a phage-selected zinc finger fused to the activation domain of HSV VP 16. and a reporter plasmid which contains the recognition sequence of the zinc finger upstream of a CAT reporter gene.

Thus, a zinc finger which recognises the DNA sequence AAAAAAGGCG is selected by phage display as described in Example 1. By the method of the preceding examples said zinc finger is used to construct transcription factors as described above.

A transient expression experiment is conducted, wherein the CAT reporter gene on the reporter plasmid is placed downstream of the sequence AAAAAAGGCG. The reporter plasmid is cotransfected with a plasmid vector expressing the zinc finger-HSV fision under the control of a constitutive promoter. No activation of CAT gene expression is observed.

However, when the same experiment is conducted in the presence of Distamycin A. CAT expression is observed as a result of the binding of the zinc finger transcription factor to its recognition sequence AAAAAAGGCG.

### Example 5

### Isolation of cognate target nucleic acids

Using a known DNA binding molecule, target DNA sequences to which it can bind are isolated. The 434 repressor is a gene regulatory protein of phage 434. It binds to a 1 4bp operator site (see Koudelka et al. 1987. Nature vol 326 pp 886-888). This operator site consists of five conserved bp (1-5), then four variable bp (6-9), then five more conserved bp (10-14) as shown below:

Site: 1 5 6 7 8 9 10 14

Base: A C A A G/T X X X X A/T T T G T

wherein X is any base.

The conserved bases contact the 434 repressor protein. The four variable bases are thought not to contact the 434 repressor protein. However, the four bases which do not contact the 434 repressor protein may affect the affinity of binding of the repressor to the operator site.

The 434 repressor protein (i.e., the DNA binding molecule) is contacted with a library of different target DNA sequences in the presence and absence of ligand:

The target DNA sequences are synthesized using an Applied Biosystems 380A DNA synthesizer and are purified by gel electrophoresis. The four variable bases (‘X’ as shown above) are randomised, producing a library of 256 different target DNA molecules. position 5 being T, and position 10 being A. At the 5′ and 3′ ends of this sequence are placed PCR primer sequences for amplification and recovery of the central target sequences.

Structure of Target DNA Sequence Library:

5′ 1 6 9 14
GTCGGATCCTGTCTGAGGTGAGACAATXXXXATTGGTCTTCCGACGTCGA

3′
ATTCGCG

wherein X is any base, and the partially randomised 434 operator is underlined.

The 434 repressor protein is added to the library of target DNA sequences, in the presence and absence of 2 μM distamycin A (Sigma) ligand in 200 μl binding buffer (9 μM Tris-HCl pH 8.0, 90 mM KCl. 90 μM ZnSO₄) and incubated for 30 min.

Nitrocellulose filters (BA 85. Schleicher and Schtiil) are placed into a suction chamber (as in Thiesen et al. (eds), Immunological Methods vol IV. Academic Press, Orlando) and prewet with 600 ml Tris-HCl binding buffer. The protein-oligonucleotide mix is applied to the filter(s) with gentle suction, the filters are washed with 4 ml Tris-HCl binding buffer. Oligonucelotides are eluted in 200 μl binding buffer plus 1 mM 1-10-o-phenanthroline.

Oligonucleotides are then amplified by PCR, using the following primers:

| | |
|---|---|
| Primer A | 5′-GCGGATCCTGTCTGAGGTGAG-3′ |
| Primer B | 5′-CGCGAATTCGACGTCGGAAGAC-3′ |

using an amplification kit (Perkin Elmer Cetus) with the following cycling regime: 93° C. 30 sec; 45° C. 120 sec; 45° C, to 67° C., ramp 60 sec; 67° C. 180 sec for 25 cycles. 1 μl of eluted oligonucleotide material is used as template.

Optionally, the PCR amplified DNA product is then used in further rounds of incubation with the 434 repressor protein, nitrocellulose filter binding, oligonucleotide elution and PCR amplification.

PCR amplified DNA products are then sequenced using standard techniques.

Target DNA sequences are selected which bind the 434 repressor with higher affinity in the presence of ligand than in the absence of ligand. Furthermore, DNA sequences are selected which bind the 434 repressor in the absence of ligand with a higher affinity than in the presence of ligand.

### Example 6

### Isolation of ligands Which Affect the Binding of a DNA Binding Molecule to Its Cognate DNA Target

The 434 repressor protein of Example 5 is used in conjunction with a target operator DNA sequence to which it binds.

The operator sequence used is

5′-A C A A T A A A T A T G T-3′

A library of ligands is used in place of the 2 μM distamycin A (Sigma) ligand of Example 5.

ligands are isolated which are capable of increasing the affinity of the 434 repressor for its cognate DNA target sequence, ligands are also isolated which are capable of decreasing the affinity of the 434 repressor for its cognate DNA target sequence.

### Example 7

### Generation of Transgenic Plants Expressing a Zinc Finger Protein Fused to a Transactivation Domain

To investigate the utility of heterologous zinc finger proteins for the regulation of plant genes, a synthetic zinc finger protein was designed and introduced into transgenic Arabidopsis thaliana under the control of a promoter capable of expression in a plant as described below. A second construct comprising the zinc finger protein binding sequence fused upstream of the Green Fluorescent Protein (GFP) reporter gene was also introduced into transgenic Arabidopsis thaliana as described in Example 8. Crossing the two transgenic lines produced progeny plants carrying both constructs in which the GFP reporter gene was expressed demonstrating transactivation of the gene by the zinc finger protein.

Using conventional cloning techniques, the following constructs were made as Xbal-BamHI fragments in the cloning vector pcDNA3.1 (Invitrogen).

pTFIILIAZifVP16

pTFIIIAZifVP 16 comprises a fusion of four finger domains of the zinc finger protein TFIIIA fused to the three fingers of the zinc finger protein Zif68. The TFIIIA-derived sequence is fused in frame to the translational initiation sequence ATG. The 7 amino acid Nuclear Localisation Sequence (NLS) of the wild-type Simian Virus 40 Large T-Antigen is fused to the 3 end of the ZiD68 sequence, and the VP1 6 transactivation sequence is fused downstream of the NLS. In addition, 30 bp sequence from the c-myc gene is introduced downstream of the VP16 domain as a “tag” to facilitate cellular localisation studies of the trangene. While this is experimentally useful, the presence of this tag is not required for the activation (or repression) of gene expression via zinc finger proteins.

The sequence of pTFIIIAZifVP16 is shown in SEQ ID No. 1 as an XbaI-BamHI fragment. The translational initiating ATG is located at position 15 and is double underlined. Fingers 1 to 4 of TFIIIA extend from position 18 to position 416. Finger 4 (positions 308-416) does not bind DNA within the target sequence, but instead serves to separate the first three fingers of TFLUA from Zif68 which is located at positions 417-689. The NLS is located at positions 701-722, the VP16 transactivation domain from positions 723-956. and the c-mvc tag from positions 957-986. This is followed by the translational terminator TAA.

pTFIIIAZifVP64

pTFIIIAZifVP64 is similar to pTFIIIAZifVP16 except that the VP64 transactivation sequence replaces the VP 16 sequence of pTFIIIAZifVP16.

The sequence of pTFIIIAZifVP64 is shown in SEQ ID No. 2 as an XbaI-BamHI fragment. Locations within this sequence are as for pTFIIIAZifvP 1 6 except that the VP64 domain is located at position 723-908 and the c-myc tag from positions 909-938.

Using conventional cloning, techniques, the sequence 5′-AAGGAGATATAACA-3′ is introduced upstream of the translational initiating ATG of both pTFIIIAZifVP 16 and pTFIIIAZifVP64. This sequence incorporates a plant translational initiation context sequence to facilitate translation in plant cells (Prasher et al. Gene 11: 229-233 (1992); Chalfie et al. Science 263 : 802-805 (1992)).

The final constructs are transferred to the plant binary vector pBIN 12 1 between the Cauliflower Mosaic Virus 315S promoter and the nopaline synthase terminator sequence. This transfer is effected using the XbaI site of PBIN 121. The binary constructs thus derived are then introduced into Agrobacterium tumefaciens (strain LBA 4044 or GV1101) either by triparental mating or direct transformation.

Next, Arabidopsis thaliana are transformed with Agrobacterium containing the binary vector construct using conventional transformation techniques. For example, using vacuum infiltration (e.g. Bechtold et al. CR Acad Sci Paris 31160: 1194-1199. Bent et al. Science 265: 1856-1860 (1994)), transformation can be undertaken essentially as follows. Seeds of Arabidopsis are planted on top of cheesecloth covered soil and allowed to grow at a final density of 1 per square inch under conditions of 16 hours light/8 hours dark. After 4-6 weeks, plants are ready to infiltrate. An overnight liquid culture of Agrobacterium carrying the appropriate construct is grown up at 28° C, and used to inoculate a fresh 500 ml culture. This culture is grown to an OD₆₀₀ of at least 2.0, after which the cells are harvested by centrifugation and resuspended in 1 liter of infiltration medium (1 liter prepared to contain: 2.2 gMS Salts, 1× B5 vitamins, 50 g sucrose. 0.5 g MES pH 5.7, 0.044 μM benzylaminopurine, 200 L Silwet 11L-77 (OSI Specialty)). To vacuum infiltrate. pots are inverted into the infiltration medium and placed into a vacuum oven at room temperature. Infiltration is allowed to proceed for 5 mins at 400 mm Hg. After releasing the vacuum, the pot is removed and layed it on its side and covered with Saran wrap. The cover is removed the next day and the plant stood upright. Seeds harvested from infiltrated plants are surface sterilized and selected on appropriate medium. Vernalizalizion is undertaken for two nights at around 4° C. Plates are then transferred to a plant growth chamber. After about 7 days, transformants are visible and are transferred to soil and grown to maturity.

Many transgenic plants are grown to maturity. They appear phenotypically normal and are selfed to homozygosity using standard techniques involving crossing and germination of progeny on appropriate concentration of antibiotoic.

Transgenic plant lines carrying the TFIIIAZifVP 16 construct are designated At-TFIIIAZifVP16 and transgenic plant lines carrying the TFIIIAZifVP64 construct are designated At-TFIIIAZifVP64.

### Example 8

### Generation of Transgenic Plants Carrying a Green Fluorescent Protein Reporter Gene

A reporter plasmid is constructed which incorporates the target DNA sequence of the TFIIIAZifVP 1-6 and TFIIIAZifVP64 zinc finger proteins described above upstream of the Green Fluorescent Protein (GFP) reporter gene. The target DNA sequence of TFIIIAZifVP16 and TFIIIAZifVP64 is shown in SEQ I.D. No. 3. This sequence is incorporated in single copy immediately upstream of the CAMV 35S -90 minimal promoter to which the GFP gene is fused.

The resultant plasmid, designated pTFIIIAZif-UAS/GFP, is transferred to the plarit binary vector pBINI21 replacing the Cauliflower Mosaic Virus D5S promoter. This construct is then transferred to Agrobacterium tumefaciens and subsequently transferred to Arabidopsis thaliana as described above. Transgenic plants carrying the construct are designated At-TFIIIAZif-UAS/GFP.

### Example 9

### Use of Zinc Finger Proteins to Up-Regulate a Transgene in a Plant

To assess whether the zinc finger constructs TFHIIAZifVP 16 and TFIIIAZifVP64 are able to transactivate gene expression in planta, Arabidopsis lines A4-TFIIIAZifVP 16 and At-TFIIIAZifVP64 are crossed to At-TfIIIAZif-UAS/GFP. The progeny of such crosses yield plants that carry the reporter construct TFIIIZif-UAS/GFP together with either the zinc finger protein construct TFIIIAZifVP16 or the zinc finger construct TFIIIAZifVP64.

Plants are screened for GFP expression using an inverted fluorescence microscope (Leitz DM-EL) fitted with a filter set (Leitz-D excitation BP 355425, dichronic 455, emission LP 460) suitable for the main 395 nm excitation and 509 nm emission peaks of GFP.

In each case, the zinc finder construct is able to transactivate gene expression demonstrating the utility of heterologous zinc finger proteins for the regulation of plant genes.

### Example 10

### Generation of Transgenic Plants Expressing a Zinc Finger Fused to a Plant Transactivation Domain

The constructs pTFIIAZifVP16 and pTFIIIAZifVP64 utilise the VP16 and VP64 transactivation domains of Herpes Simplex Virus to activate gene expression. Alternative transactivation domains are various and include the C1 transactivation domain sequence (from maize; see Goffet al.; Genes Dev. 5: 298-309 (1991); Goffet al.; Genes Dev. 6: 864-875 (1992)), and a number of other domains that have been reported from plants (see Estruch et al.; Nucl. Acids Res. 22: 3983-3989 (1994)).

Construct pTFIIAZifC1 is made as described above for pTFIIIAZifVP16 and pTFIIIAZifVP64 except the VP16/VP64 activation domains are replaced with the C1 transactivation domain sequence

A transgenic Arabidopsis line, designated At-TFIIIAZifC1, is produced as described above in Example 8 and crossed with At-TFIIIAZif-UAS/GFP. The progeny of such crosses yield plants that carry the reporter construct TFIIIAZif-UAS/GFP together with either the zinc finger protein construct TFIIIAZifC1.

Plants are screened for GFP expression using an inverted fluorescence microscope (Leitz DM-IL) fitted with a filter set (Leitz-D excitation BP 355425. dichronic 455, emission LP 460) suitable for the main 395 nm excitation and 509 nm emission peaks of GFP.

### Example 11

### Regulation of an Endogenous Plant Gene—UDP Glucose Flavonoid Glucosyl-Transferase (UFGT).

To determine whether a suitably configured zinc finger could be used to regulate gene transcription from an endogenous gene in a plant, the maize UDP glucose flavonoid glucosyl-transferase (UFGT) gene (the Bronze1 gene) was selected as the target gene. UFGT is involved in anthocyanin biosynthesis. A number of wild type alleles have been identified including Bz-W22 that conditions a purple phenotypes in the maize seed and plant. The Bronze locus has been the subject of extensive genetic research because its phenotype is easy to score and its expression is tissue specific and varied (for example aleurone, anthers, husks, cob and roots). The complete sequence of Bz-W22 including upstream regulatory sequences has been determined (Ralston et al., Genetics 119: 185-197). A number of sequence motifs that bind transcriptional regulatory proteins have been identified within the Bronze promoter including sequences homologous to consensus binding sites for the myb- and myc-like proteins (Roth et al., Plant Cell 3: 317-325).

Identification of a Zinc Finger That Binds to the Bronze Promoter

The first step is to carry out a screen for zinc finger proteins that bind to a selected region of the Bronze promoter. A region is chosen just upstream of the AT rich block located at between −88 and −80, which has been shown to be critical for Bz1 expression (Roth et al., supra).

1. Bacterial colonies containing phage libraries that express a library of zinc fingers randomised at one or more DNA binding residues (see Example 1) are transferred from plates to culture medium. Bacterial cultures are grown overnight at 30° C. Culture supernatant containing phases is obtained by centrifuganon.

2. 10 μmol of biotinylated target DNA, derived from the Bronze promoter. immobilised on 50 mg streptavidin beads (Dynal) is incubated with 1 ml of the bacterial culture supernatant diluted 1:1 with PBS containing 50 μM ZnCl₂, 4% Marvel. 2% Tween in a streptavidin coated tube for 1 hour at 20° C, on a rolling platform in the presence of 4 μg poly [d(I−C)] as competitor.

3. The tubes are washed 20 times with PBS containing 50 μM ZnCl₂ and 1% Tween, and 3 times with PBS containing 50 μM ZnCl₂ to remove non-binding phage.

4. The remaining phage are eluted using 0.1 ml 0.1 M triethylamine and the solution is neutralised with an equal volume of 1 M Tris-Cl (pH 7.4).

5. Logarithmic-phase E. coli TG1 cells are infected with eluted phage, and grown overnight, as described above, to prepare phage supernatants for subsequent rounds of selection.

6. Single colonies of transformants obtained after four rounds of selection (steps 1 to 5) as described, are grown overnight in culture. Single-stranded DNA is prepared from phage in the culture supernatant and sequenced using the Sequenase12.0 kit U.S. Biochemical Cord.). The amino acid sequences of the zinc finger clones are deduced.

Construction of a Vector for Expression of the Zinc Finger Clone Fused to a C1 Activation Domain in Maize Protoplasts

Using conventional cloning techniques and in a similar manner to Example 7, the construct pZifBz23C1 is made in cloning vector pcDNA3.I (Invitrogen).

pZifBf23C1 comprises the three fingers of the zinc finger protein clone ZifBz23s fused in frame to the translational initiation sequence ATG. The 7 amino acid Nuclear Localisation Sequence NS) of the wild-type Simian Virus 40 Large T-Antigen is fused to the 3′ end of the ZifBz23 sequence, and the C1 transaction sequence is fused downstream of the NLS. In addition, 30 bp sequence from the c-myc gene is introduced downstream of the VP16 domain as a “tag” to facilitate cellular localisation studies of the trangene.

The coding sequences of pZifB713C1 are transferred to a plant expression vector suitable for use in maize protoplasts, the coding sequence being under the control of a constitutive CaMV 35S promoter. The resulting plasmid is termed pTMBz23. The vector also contains a hygromycin resistance gene for selection purposes.

A suspension culture of maize cells is prepared from calli derived from embryos obtained from inbred W22 maize stocks grown to flowering in a greenhouse and self pollinated using essentially the protocol described in EP-A-332104 (Examples 40 and 41). The suspension culture is then used to prepare protoplasts using essentially the protocol described in EP-A-3-32104 (Example 42).

Protoplasts are resuspended in 0.2 M mannitol, 0.1% wly MES. 72 mM NaCl, 70 mM CaCl₂, 2.5 mM KCl, 2.5 mM glucose pH to 5.8 with KOH, at a density of about 2×10⁶ per ml. 1 ml of the protoplast suspension is then aliquotted into plastic electroporation cuvettes and 10 μg of linearized pTBz23 added. Electroporation is carried out s described in EP-A-3 32104 (Example 57). Protoplasts are cultured following transformation at a density of 2×10⁶ per ml in KM-8p medium with no solidifying agent added.

Measurements of the levels UFGT expression are made using colorimetry and/or biochemical detection methods such as Northern blots or the enzyme activitv assays described by Dooner and Nelson, Proc. Natl. Acad. Sci. 74: 5623-5627 (1977). Comparison is made with mock treated protoplasts transformed with a vector only control.

Alternatively, or in addition to, analysing expression of UFGT in transformed protoplasts, intact maize plants may be recovered from transformed protoplasts and the extent of UFGT expression determined. Suitable protocols for growing up maize plants from transformed protoplasts are known in the art: Electroporated protoplasts are resuspended in Km-8p medium containing 1.2% w/v Seaplaque agarose and 1 mg/l 2.4-D.

Once the gel has set, protoplasts in agarose are place in the dark at 26° C. After 14 days, clonies arise from the protoplasts. The agarose containing the colonies is transferred to the surface of a 9 cm diameter petri dish containing 30 ml of N6 medium (EP-A-332.104) containing 9.4-D solidified with 0.24% Gelrite(D. 100 mg/l hydromycin B is also added to select for transformed cells. The callus is cultured further in the dark at 26° C, and callus pieces subcultured every two weeks onto fresh solid medium. Pieces of callus may be analysed for the presence of the pTMBz′3 construct and/or UFGT expression determined.

Corn plants are regenerated as described in Example 47 of EP-A-332.104. Plantlets appear in 4 to 8 weeks. When 2 cm tall, plantlets are transferred to ON6 medium (EP-A-332,104) in GA7 containers and roots form in 2 to 4 weeks. After transfer to peat pots plants soon become established and can then be treated as normal corn plants.

Plantlets and plants can be assayed for UFGT expression as described above.

### Example 12

### Regulation of Gene Expression Using a Chemically Inducible Small Molecule

The Zif268 Zinc finger phage display library described in Example 1 is screened using the bronze promoter sequence described in Example 11 and a library of small molecule candidate ligands, pre-screened to remove non-DNA binding molecules. The protocol used is essentially a modification of Example 1 but using multiple ligands. To increase the number of ligands in the screen, ligands are screened in groups of twenty. Once zinc finger clones are identified that have ligand-dependent DNA binding, a single zinc finger clones is tested for ligand-dependent binding against each individual ligand in the mixture originally selected. In this way, a gene switch comprising a zinc finger clone that binds to a region of the bronze promoter in a manner modulatable by a chemical ligand, the region of the bronze promoter and the chemical ligand itself is identified.

The zinc finger clone is fused to a VP16 transactivation domain and other relevant sequences as described in Example 7. The resulting construct, pZFSelectC1 is transferred to the plant binary vector pBIN121 between the Cauliflower Mosaic Virus 35S promoter and the nopaline synthase terminator sequence. The binary construct thus derived is then introduced into Agrobacterium tumefaciens (strain LBA 4044 or GV101) either by triparental mating or direct transformation.

A transgenic Arabidopsis line, designated At-ZFSelectC1, is produced as described above in Example 8.

A further transgenic Arabidopsis line, designated At-BzGUS is produced which comprises a reporter construct containing the E. coli beta-glucuronidase gene (GUS) fused to a −90 minimal 35S promoter to which is operably linked the bronze promoter sequence used in the tripartite screen. Arabidopsis lacks endogenous GUS activity. Further. GUS activity is very stable and expression can be measured accurately using flurometric assays of very small amounts of transformed plant tissue (see Jefferson et al. Embo J. 6: 3901-3907(1987)).

At-ZFSelectC1 lines are crossed with At-BzGUS lines. The progeny of such crosses yield plants that carry the reporter construct BzGUS together with either the zinc finger protein construct ZFSelectC1.

Plants are grown in a range of concentrations of the chemical ligand and GUS activitv in leaf tissue measured as described in Jefferson et al., Embo J. 6: 3901-3907 (1987). GUS activity in non transgenic plants. At-ZFSelectC1 line and At-BzGUS lines in the presence of the chemical ligand is also measured.

### Example 13

### Tripartite Screen for a Zinc Finger/Target DNA and Small Molecule Ligand and the Use of the Identified Components in Regulating Gene Expression

A screen is performed as described in Example 12 except that the target DNA is a randomised library based on the Bronze promoter sequence and the procedure described in Example 1.3 is used to determine the binding site signature of identified clones once a ligand has been selected. Verification of the target DNA sequence is also performed as described in Example 1.3.

A target DNA identified in the screen is introduced into a −90 minimal Ca35S-GUS reporter construct as described in Example 12 and used to produce a transgenic Arabidopsis line. A corresponding zinc finger clone is introduced into an expression construct as described in Example 12 and used to produce a transgenic Arabidopsis line. The two lines are crossed and progeny tested for induction of GUS activity in the presence or absence of the ligand identified in the screen.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

```
Sequence ID 1:TFIIIA/Zif-VP16
          TCTAGAGCGCCGCCATGGGAGAGAAGGCGCTGCCGGTGGTGTATAAGCGG
TACATCTGCTCTTTCGCCGACTGCGGCGCTGCTTATAACAAGAACTGGAA
ACTGCAGGCGCATCTGTGCAAACACACAGGAGAGAAACCATTTCCATGTA
AGGAAGAAGGATGTGAGAAAGGCTTTACCTCGCTTCATCACTTAACCCGC
CACTCACTCACTCATACTGGCGAGAAAAACTTCACATGTGACTCGGATGG
ATGTGACTTGAGATTTACTACAAAGGCAAACATGAAGAAGCACTTTAACA
GATTCCATAACATCAAGATCTGCGTCTATGTGTGCCATTTTGAGAACTGT
GGCAAAGCATTCAAGAAACACAATCAATTAAAGGTTCATCAGTTCAGTCA
CACACAGCAGCTGCCGTATGCTTGCCCTGTCGAGTCCTGCGATCGCCGCT
TTTCTCGCTCGGATGAGCTTACCCGCCATATCCGCATCCACACAGGCCAG
AAGCCCTTCCAGTGTCGAATCTGCATGCGTAACTTCAGTCGTAGTGACCA
CCTTACCACCCACATCCGCACCCACACAGGCGAGAAGCCTTTTGCCTGTG
ACATTTGTGGGAGGAAGTTTGCCAGCAGTGATGAACGCAAGAGGCATACC
AAAATCCATTTAAGACAGAAGGACGCGGCCGCACTCGAGCGGAATTCCGG
CCCAAAAAAGAAGAGAAAGGTCGCCCCCCCGACCGATGTCAGCCTGGGGG
ACGAGCTCCACTTAGACGGCGAGGACGTGGCGATGGCGCATGCCGACGCG
CTAGACGATTTCGATCTGGACATGTTGGGGGACGGGGATTCCCCGGGGCC
GGGATTTACCCCCCACGACTCCGCCCCCTACGGcGCTCTGGATACGGCCG
ACTTCGAGTTTGAGCAGATGTTTACCGATGCCCTTGGAATTGACGAGTAC
GGTGGGGAACAAAAACTTATTTCTGAAGAAGATCTGTAAGGATCC
Sequence ID 2: TFIIIA/Zif-VP64
          TCTAGAGCGCCGCCATGGGAGAGAAGGCGCTCCCGGTGGTGTATAAGCGG
TACATCTGCTCTTTCGCCGACTGCGGCGCTGCTTATAACAAGAACTGGAA
ACTGCAGGCGCATCTGTGCAAACACACAGGAGAGAAACCATTTCCATGTA
AGGAAGAAGGATGTGAGAAAGGCTTTACCTCGCTTCATCACTTAACCCGC
CACTCACTCACTCATACTGGCGAGAAAAACTTCACATGTGACTCGGATGG
ATGTGACTTGAGATTTACTACAAAGGCAAACATGAAGAAGCACTTTAACA
GATTCCATAACATCAAGATCTGCGTCTATGTGTGCCATTTTGAGAACTGT
GGCAAAGCATTCAAGAAACACAATCAATTAAAGGTTCATCAGTTCAGTCA
CACACAGCAGCTGCCGTATGCTTGCCCTGTCGAGTCCTGCGATCGCCGCT
TTTCTCGCTCGGATGAGCTTACCCGCCATATCCGCATCCACACAGGCCAG
AAGCCCTTCCAGTGTCGAATCTGCATGCGTAACTTCAGTCGTAGTGACCA
CCTTACCACCCACATCCGCACCCACACAGGCGAGAAGCCTTTTGCCTGTG
ACATTTGTGGGAGGAAGTTTGCCAGGAGTGATGAACGCAAGAGGCATACC
AAAATCCATTTAAGACAGAAGGACGCGGCCGACTCGAGCGGAATTCCGGC
CCAAAAAAGAAGAGAAAGGTCGAACT
```

1. A method of selecting a switching system, the switching system comprising: (i) a first component comprising a first molecule and (ii) a second component comprising a second molecule, in which the first molecule binds to the second molecule in a manner modulatable by a ligand, and (iii) a third component comprising the ligand. the method comprising the steps of:
(a) contacting one or more candidate first molecules with one or more candidate second molecules in the presence of one or more ligands;
(b) selecting a complex of the three components;
(c) optionally isolating and/or identifying the unknown components of the complex;
(d) comparing the binding of the first molecule component of the complex to the second molecule component of the complex in the presence and absence of the Fields 15 ligand component of the complex; and
(e) selecting complexes where said binding differs in the presence and absence of the ligand component,
in which at least one component is provided in the form of a library of members.

2. A method according to claim 1, in which at least one of the candidate first molecules comprises a non-naturally occurring binding domain which binds to the second molecule.

3. A method according to claim 1 or 2, in which one of the first molecule and second molecule comprises a nucleic acid binding molecule, and the other of the first and second molecules comprises a nucleic acid.

4. A method according to claim 1, 2 or 3, in which one or both of the candidate nucleic acid and nucleic acid binding molecules is provided as a plurality of molecules.
TCAGCTGACTTCGGATGCATTAGATGACTTTGACTTAGATATGCTAGGAT
CTGACGCGCTAGACGATTTCGATCTGGACATGTTGGGCAGCGATGCTcTG
GACGATTTCGATTTAGATATGCTTGGCTCGGATGCCCTGGATGACTTCGA
CCTCGACATGCTGTCAAGTCAGCTGAGCCAGGAACAAAAACTTATTTCTG
AAGAAGATCTGTAAGGATCC
Sequence ID 3: TFIIIA/Zif binding site
TgcgcgggcgTGTACCTggatgggagacC

5. A method according to claim 4, in which the nucleic acid binding molecule is provided as a library of nucleic acid binding molecules.

6. A method according to claim 4 or 5, in which a single target nucleic acid is used.

7. A method according to claim 4, 5 or 6, in which one of the components isolated and/or identified in step (c) is a ligand component.

8. A method according to any of claims 4 to 7, in which one of the components isolated in step (c) is a nucleic acid binding molecule component.

9. A method according to any of claims 4 to 8, in which the nucleic acid is provided as a library of nucleic acid sequences, the sequences being related to one another by sequence homology.

10. A method according to any of claims 4 to 9, in which a plurality of candidate ligands are used.

11. A method according to any of claims 4 to 10, in which the ligands are provided as a library of ligands.

12. A method according to any of claims 4 to 11, in which the candidate nucleic acid binding molecules are polypeptides.

13. A method according to any of claims 4 to 12, in which the polypeptides are at least-partly derived from DNA binding proteins, preferably transcription factors.

14. A method according to any of claims 4 to 12, in which the candidate nucleic acid binding molecules are derived from zinc finger transcription factors.

15. A method according to any of claims 4 to 13, in which the candidate nucleic acid binding molecules are provided as a phage display library.

16. A method according to any of claims 4 to 14, in which the ligand is selected from Distamycin A, Actinomycin D and echinomycin.

17. A switching system comprising a gene switch, in which the switching system has been selected by a method according to any of claims 4 to 15.

18. Use of a nucleic acid binding molecule selected by a method according to any one of claims 4 to 15 in a method of regulating transcription from a nucleic acid sequence comprising a target nucleic acid to which the nucleic acid binding molecule binds in a manner modulatable by a ligand.

19. Use of a ligand selected by a method according to any one of claims 4 to 15 in a method of regulating transcription from a nucleic acid sequence comprising a target nucleic acid to which a nucleic acid binding molecule binds in a manner modulatable by the ligand.

20. Use of a target nucleic acid selected by the method of any one of claims 4 to 15 in a method of regulating transcription from a nucleic acid sequence comprising the target nucleic acid to which a nucleic acid binding molecule binds in a manner modulatable by a ligand.

21. A method of modulating the expression of one or more genes, said method comprising administering a nucleic acid binding molecule and a ligand selected according to the method of any one of claims 4 to 15 to a cell, in which the regulatory sequences of the genes comprise a target nucleic acid selected according to a method according to any of claims 4 to 15.

22. A method of modulating the expression of one or more nucleotide sequences of interest in a host cell which host cell comprises a nucleic acid sequence capable of directing the expression of a nucleic acid binding molecule and a target nucleic acid sequence to which the nucleic acid binding molecule binds in a manner modulatable by a ligand, which method comprises administering said ligand to the cell and wherein the nucleic acid binding molecule is heterologous to the host cell.

23. A method according to claim 21 or 22 wherein the host cell is a plant cell.

24. A method according to claim 23, in which the plant cell is part of a plant and the target sequence is part of a regulatory sequence to which the nucleotide sequence of interest is operably linked, said regulatory sequence being preferentially active in the male or female organs of the plant.

25. A non human transgenic organism comprising a target nucleic acid sequence and a nucleic acid sequence capable of directing the expression of a nucleic acid binding molecule which binds to the target nucleic acid in a manner modulatable by a ligand, in which the target nucleic acid sequence and/or nucleic acid sequence are heterologous to the organism.

26. A transgenic non-human organism according to claim 25, which is a plant.

27. A method according to claim 1 or 2, in which each of the first and second molecules comprises a polypeptide.

28. A method according to claim 27, in which the first molecule comprises a polypeptide binding protein and the second molecule comprises a polypeptide.

29. A method according to claim 27 or 28, in which one or both of the first and second molecules is provided as a library of polypeptides.

30. A method according to claim 27, 28 or 29, in which the ligands are provided as a library of ligands.

31. A method according to any of claims 27 to 30, in which the ligand is an immunoglobulin molecule, preferably an antibody molecule.

32. A method according to any of claims 27 to 31, in which the first molecule is a nucleic acid binding protein capable of binding to nucleic acid.

33. A method according to claim 32, in which the nucleic acid binding protein binds to nucleic acid in a manner modulatable by the second molecule.

34. A switching system comprising a protein switch, in which the switching system has been selected by a method according to any of claims 27 to 33.

35. Use of a nucleic acid binding protein selected by a method according to claim 33, in a method of regulating transcription from a nucleic acid sequence comprising a target nucleic acid to which the nucleic acid binding protein binds.

36. Use of a ligand selected by a method according to claim 33, in a method of regulating transcription from a nucleic acid sequence comprising a target nucleic acid to which the nucleic acid binding protein binds in a manner modulatable by the ligand.

37. Use of a target nucleic acid selected by the method according to claim 33, in a method of regulating transcription from a nucleic acid sequence comprising a target nucleic acid to which the nucleic acid binding protein binds in a manner modulatable by a ligand.

38. A method of modulating the expression of one or more genes, said method comprising administering a nucleic acid binding protein and a ligand selected according to a method according to claim 33 to a cell, in which the regulatory sequences of the genes comprise a target nucleic acid to which the nucleic acid binding protein binds in a manner modulatable by a ligand.

39. A method of modulating the expression of one or more nucleotide sequences of interest in a host cell which host cell comprises a first nucleic acid sequence capable of directing the expression of a nucleic acid binding protein, a second nucleic acid sequence capable of directing the expression of a second polypeptide, the binding between the nucleic acid binding to the second polypeptide being modulatable by a ligand, and a target nucleic acid sequence to which the nucleic acid binding protein binds in a manner modulatable by a second polypeptide which method comprises administering said ligand to the cell.

40. A method according to claim 39, in which the nucleic acid binding protein is heterologous to the host cell.

41. A method according to claim 39 or 40 wherein the host cell is a plant cell.

42. A method according to claim 42, in which the plant cell is part of a plant and the target sequence is part of a regulatory sequence to which the nucleotide sequence of interest is operably linked, said regulatory sequence being preferentially active in the male or female organs of the plant.

43. A non human transgenic organism comprising a target nucleic acid sequence, a first nucleic acid sequence capable of directing the expression of a nucleic acid binding protein, and a second nucleic acid sequence capable of directing the expression of a second polypeptide which binds to the nucleic acid binding protein in a manner modulatable by a ligand, in which the nucleic acid binding protein binds to the target nucleic acid sequence in a manner modulatable by binding of the second polypeptide.

44. A method according to claim 43, in which any or all of the first nucleic acid sequence, the second nucleic acid sequence, and the target nucleic acid sequence are heterologous to the organism.

45. A transgenic non-human organism according to claim 43 or 44 which is a plant.

46. A method according to any of claims 1 to 16 and 27 to 33, in which the first molecule component of the complex has a higher affinity for the second molecule component of the complex in the presence of the ligand component than in the absence of the ligand component.

47. A method according to of claims 1 to 16 and 27 to 33, in which the first molecule component of the complex has a higher affinity for the second molecule component of the complex in the absence of the ligand component than in the presence of the ligand component.
